SEQUENCE LISTING (30 sequences)

SEQ ID NO: 1 - protein, 11 aa, Artificial Sequence
  Artificial collagen sequence
LTGPIGPPGPA

SEQ ID NO: 2 - protein, 33 aa, Artificial Sequence
  Artificial collagen sequence
GAPGKDGVRGLTGPIGPPGPAGAPGDKGEAGPS

SEQ ID NO: 3 - protein, 24 aa, Artificial Sequence
  Artificial collagen sequence
GAPGKDGVRGLTGPIGPPGPAGAP

SEQ ID NO: 4 - protein, 24 aa, Artificial Sequence
  Artificial collagen sequence
GAPGKDGVRGLTGPIGPPGPAGAR

SEQ ID NO: 5 - protein, 24 aa, Artificial Sequence
  Artificial collagen sequence
GRPGPPGPPGARGQAGVMGFPGPK

SEQ ID NO: 6 - protein, 11 aa, Artificial Sequence
  Artificial collagen sequence
  MOD_RES (8)..(8): 4Hyp
LTGPIGPPGPA

SEQ ID NO: 7 - protein, 33 aa, Artificial Sequence; identical to SEQ ID NO: 2
  Artificial collagen sequence
  MOD_RES (3)..(3): 4Hyp
  MOD_RES (18)..(18): 4Hyp
  MOD_RES (24)..(24): 4Hyp

SEQ ID NO: 8 - protein, 24 aa, Artificial Sequence
  Artificial collagen sequence
  MOD_RES (3)..(3): 4Hyp
  MOD_RES (18)..(18): 4Hyp
  MOD_RES (24)..(24): 4Hyp
GAPGKDGVRGLTGPIGPPGPAGAP

SEQ ID NO: 9 - protein, 24 aa, Artificial Sequence
  Artificial collagen sequence
  MOD_RES (3)..(3): 4Hyp
  MOD_RES (18)..(18): 4Hyp
GAPGKDGVRGLTGPIGPPGPAGAR

SEQ ID NO: 10 - protein, 24 aa, Artificial Sequence
  Artificial collagen sequence
  MOD_RES (3)..(3): 4Hyp
  MOD_RES (9)..(9): 4Hyp
  MOD_RES (21)..(21): 4Hyp
GRPGPPGPPGARGQAGVMGFPGPK

SEQ ID NO: 11 - protein, 11 aa, Artificial Sequence
  Artificial collagen sequence
GAPGKDGVRGL

SEQ ID NO: 12 - protein, 23 aa, Artificial Sequence
  Artificial collagen sequence
GAPGKDGVRGLTGPIGPPGPAGA

SEQ ID NO: 13 - protein, 58 aa, Artificial Sequence
  Artificial collagen sequence
GAPGKDGVRGLTGPIGPPGPAGAPGDKGEAGPSGPAGPTGARGAPGDRGEPGPPGPAG

SEQ ID NO: 14 - protein, 90 aa, Artificial Sequence
  Artificial collagen sequence
GAPGKDGVRGLTGPIGPPGPAGAPGDKGEAGPSGPAGPTGARGAPGDRGEPGPPGPAGFAGPPGADGQPGAKGEPGDAGAKGDAGPPGPA

SEQ ID NO: 15 - protein, 19 aa, Artificial Sequence
  Artificial collagen sequence
KGAPGADGPAGAPGTPGPQ

SEQ ID NO: 16 - protein, 35 aa, Artificial Sequence
  Artificial collagen sequence
GPPGPAGEKGAPGADGPAGAPGTPGPQGIAGQRGV

SEQ ID NO: 17 - protein, 11 aa, Artificial Sequence
  Artificial collagen sequence
  MOD_RES (3)..(3): 4Hyp
GAPGKDGVRGL

SEQ ID NO: 18 - protein, 23 aa, Artificial Sequence
  Artificial collagen sequence
  MOD_RES (3)..(3): 4Hyp
  MOD_RES (18)..(18): 4Hyp
GAPGKDGVRGLTGPIGPPGPAGA

SEQ ID NO: 19 - protein, 58 aa, Artificial Sequence; identical to SEQ ID NO: 13
  Artificial collagen sequence
  MOD_RES (3)..(3): 4Hyp
  MOD_RES (18)..(18): 4Hyp
  MOD_RES (24)..(24): 4Hyp
  MOD_RES (45)..(45): 4Hyp
  MOD_RES (51)..(51): 4Hyp
  MOD_RES (54)..(54): 4Hyp

SEQ ID NO: 20 - protein, 90 aa, Artificial Sequence; identical to SEQ ID NO: 14
  Artificial collagen sequence
  MOD_RES (3)..(3): 4Hyp
  MOD_RES (18)..(18): 4Hyp
  MOD_RES (24)..(24): 4Hyp
  MOD_RES (45)..(45): 4Hyp
  MOD_RES (51)..(51): 4Hyp
  MOD_RES (54)..(54): 4Hyp
  MOD_RES (63)..(63): 4Hyp
  MOD_RES (69)..(69): 4Hyp

SEQ ID NO: 21 - protein, 19 aa, Artificial Sequence
  Artificial collagen sequence
  MOD_RES (4)..(4): 4Hyp
  MOD_RES (13)..(13): 4Hyp
KGAPGADGPAGAPGTPGPQ

SEQ ID NO: 22 - protein, 35 aa, Artificial Sequence; identical to SEQ ID NO: 16
  Artificial collagen sequence
  MOD_RES (3)..(3): 4Hyp
  MOD_RES (12)..(12): 4Hyp
  MOD_RES (21)..(21): 4Hyp

SEQ ID NO: 23 - protein, 33 aa, Artificial Sequence
  Artificial collagen sequence
  MOD_RES (3)..(3): 4Hyp
  MOD_RES (18)..(18): 4Hyp
  MOD_RES (24)..(24): 4Hyp
GVPGKYGVRGLTGPIGPPGPLGIPGWKGFVGPT

SEQ ID NO: 24 - protein, 36 aa, Artificial Sequence
  Artificial collagen sequence
  MOD_RES (3)..(3): 4Hyp
  MOD_RES (12)..(12): 4Hyp
  MOD_RES (21)..(21): 4Hyp
GPPGPVGTKGIPGVYGPLGIPGTPGPWGILGTKRGV

SEQ ID NO: 25 - protein, 33 aa, Artificial Sequence; identical to SEQ ID NO: 2
  Artificial collagen sequence
  MOD_RES (3)..(3): 4Hyp
  MOD_RES (14)..(14): 4Hyp
  MOD_RES (17)..(17): 4Hyp
  MOD_RES (18)..(18): 4Hyp
  MOD_RES (20)..(20): 4Hyp
  MOD_RES (24)..(24): 4Hyp
  MOD_RES (32)..(32): 4Hyp

SEQ ID NO: 26 - protein, 39 aa, Artificial Sequence
  Artificial collagen sequence
  MOD_RES (9)..(9): 4Hyp
  MOD_RES (24)..(24): 4Hyp
  MOD_RES (30)..(30): 4Hyp
HHHHHHGAPGKDGVRGLTGPIGPPGPAGAPGDKGEAGPS

SEQ ID NO: 27 - protein, 33 aa, Artificial Sequence
  Artificial collagen sequence
  MOD_RES (3)..(3): 4Hyp
  MOD_RES (18)..(18): 4Hyp
  MOD_RES (24)..(24): 4Hyp
GLPGKMGVFGLTGPIGPPGPWGVPGHKGYLGPT

SEQ ID NO: 28 - protein, 33 aa, Artificial Sequence
  Artificial collagen sequence
  MOD_RES (3)..(3): 4Hyp
  MOD_RES (18)..(18): 4Hyp
  MOD_RES (24)..(24): 4Hyp
GLPGKYGVHGLTGPLGPPGPMGIPGWKGFVGPT

SEQ ID NO: 29 - protein, 35 aa, Artificial Sequence
  Artificial collagen sequence
  MOD_RES (3)..(3): 4Hyp
  MOD_RES (12)..(12): 4Hyp
  MOD_RES (21)..(21): 4Hyp
GPPGPLGMKGLPGVWGPFGLPGTPGPHGITGYKGV

SEQ ID NO: 30 - protein, 35 aa, Artificial Sequence
  Artificial collagen sequence
  MOD_RES (3)..(3): 4Hyp
  MOD_RES (12)..(12): 4Hyp
  MOD_RES (21)..(21): 4Hyp
GPPGPVGTKGLPGFYGPLGIPGHPGPWGMLGTKGV

(19) United States
(12) Patent Application Publication
(10) Pub. No.: US 2022/0073592 A1
(43) Pub. Date: Mar. 10, 2022

(54) SYNTHETIC AND RECOMBINANT COLLAGEN PEPTIDES HAVING BIOLOGICAL ACTIVITY

(71) Applicant: GELITA AG, Eberbach (DE)

(72) Inventors: Stephan Hausmanns, Heidelberg (DE); Hans-Ulrich Frech, Weinheim (DE); Steffen Oesser, Glücksburg (DE); Martin Hahn, Gronau (DE)

(21) Appl. No.: 17/416,362

(22) PCT Filed: Dec. 20, 2019
(86) PCT No.: PCT/EP2019/086613

§ 371 Date: Jun. 18, 2021

(30) Foreign Application Priority Data
Dec. 21, 2018 (DE) 10 2018 222 898.1
Feb. 26, 2019 (DE) 10 2019 202 608.7
May 28, 2019 (DE) 10 2019 207 859.1

(51) Int. Cl. C07K 14/78; A23L 33/18; A61Q 19/08; A61K 8/65

(52) U.S. Cl. CPC C07K 14/78; A23L 33/18; A61K 38/00; A61K 8/65; A61Q 19/08

(57) ABSTRACT

The present invention relates to a synthetically or recombinantly produced collagen peptide for use in a method for the therapeutic treatment of the human or animal body having a molecular weight in a range from 0.18 to 10.0 kDa, in particular 0.18 to 5.0 kDa, in particular 1.1 to 5.0 kDa, products which comprise a collagen peptide according to the invention, and a method for producing a collagen peptide according to the invention.

The present invention relates to a synthetically or recombinantly produced collagen peptide for use in a method for the therapeutic treatment of the human or animal body having a molecular weight in a range from 0.18 to 10.0 kDa, in particular 0.18 to 5.0 kDa, in particular 1.1 to 5.0 kDa, products which comprise a collagen peptide according to the invention, and a method for producing a collagen peptide according to the invention.

Collagen is an extracellular structural protein found in animals, for example, in mammals, birds, and fish. It is usually found there in the connective tissue, in particular, as part of the extracellular matrix. Tendons, ligaments, cartilage and bones are particularly rich in collagen. However, collagens are not found in plants and single-celled organisms.

Collagens occur in different, structurally and functionally different types and differ in terms of their structure, function, and origin, among other things. The polypeptide chains that make up collagen are individually synthesized in the cell on the ribosomes of the endoplasmic reticulum in the form of larger precursor molecules and have extensive repetitive (Gly-X-Y)ₙ sequences, where X and Y may be any amino acid, but usually proline and 4-hydroxyproline.

These precursor polypeptide chains are post-translationally hydroxylated on proline and lysine residues of the polypeptide chain in the endoplasmic reticulum while forming hydroxyproline and hydroxylysine residues. The hydroxylation serves to stabilize neighboring collagen polypeptide chains of the right-handed triple helix that forms in the cell, each made up of three of the precursor polypeptide chains (procollagen).

The procollagen thus formed is glycosylated intracellularly, secreted by the cell in the glycosylated triple-helical form (tropocollagen), and collagen is subsequently formed by peptidase-mediated cleavage of the terminal residues. In the course of a fibrillogenesis process, this accumulates to form collagen fibrils, which are then covalently cross-linked to form collagen fibers.

Collagen is often used in denatured form, then known as gelatin, or in the form of hydrolysates.

If gelatin and collagen are subjected to hydrolytic processes to obtain collagen peptides, in particular to enzymatic hydrolysis, collagen hydrolysates having a wide variety of compositions and application profiles may be produced, depending on the type of collagen used and the origin and the enzymatic conditions. However, these collagen hydrolysates represent a mixture of peptides, the molecular weights of which are distributed over certain size ranges. The use of such collagen hydrolysates, for example, as food supplements or as cosmetic auxiliaries, has been known for a long time, among other things, for the prevention and/or treatment of complaints that are related to the bones, the joints or to the connective tissue.

Thus, WO 2012/065782 describes collagen hydrolysates obtained from pork rind gelatin which are used to stimulate the biosynthesis of extracellular matrix proteins by skin cells and are particularly suitable for cosmetic purposes.

WO 2012/117012 discloses enzymatically hydrolyzed collagen from bovine split with an average molecular weight of 1500 to 8000 Da, which may be used together with a prebiotic for the prevention and/or treatment of osteoporosis.

Although the use of collagen hydrolysates obtained from animal materials has advantages for many applications and consumer groups, the use of collagen hydrolysates obtained in this way may also be less desirable with a view to certain consumer groups and application profiles. Certain consumer groups are fundamentally critical of or opposed to raw materials obtained from animal materials, be it that contamination including microorganisms or agents harmful to health, for example, process auxiliaries, or unwanted immune reactions are feared, or for religious or ethical reasons. In addition, the manufacturing processes used to obtain collagen hydrolysates obtained from animal materials often include complex and expensive development, purification, and further processing steps.

Against this background, it is not surprising that methods have been developed to produce gelatin, collagen, collagen hydrolysates, and individual collagen peptides by biotechnological means using recombinant genetic engineering.

Thus, WO 2006/052451 A2 discloses the production of recombinant type III collagen in Pichia pastoris strains which also express human prolylhydroxylases.

WO 2005/012356 A2 discloses the production of gelatin from human collagen type I and individual 50 kDa, 65 kDa and 100 kDa collagen peptide species, each in completely hydroxylated, partially and non-hydroxylated form.

Olsen et al. (Protein Expression and Purification, 2005, 40, pg. 346-357) discloses the recombinant production of an 8.5 kDa collagen peptide species from the al chain of human collagen in Pichia pastoris.

WO 01/34646 A2 likewise discloses the production of individual recombinant gelatin species, each with a defined molecular weight of 0 to 350 kDa resulting from the recombinant production route, which may be present in non-hydroxylated, partially or completely hydroxylated form.

The document discloses the general applicability of individual recombinant gelatin species or mixtures thereof in the food, beverage, cosmetic or pharmaceutical industries. The document discloses further possible uses of the disclosed recombinant products as a photographic composition or as a technical aid, for example in the semiconductor industry, paper manufacturing or the like.

It is therefore known to use recombinant gelatin or collagen peptides of any size individually or in a mixture, hydroxylated or non-hydroxylated, in a wide variety of technical or non-technical areas, for example in the food industry or in the therapy of human or animal diseases.

Even with a view to the increased demands in large parts of the population with regard to health, attractive outward appearance, mobility, and fitness, in each case also in advanced age, there is still a great need for food and preparations for improvement and/or the maintenance of health and treating diseases.

Health, mobility, physical attractiveness, and physical fitness are often closely related to the condition of the connective tissue. Connective tissues are tissues that occur in all tissues of the body, for example in the muscles, the intestines, the blood vessels, the gums, tendons, ligaments, bones, and the skin, and often perform supporting tasks there. Connective tissue usually has comparatively few cells and a lot of intermediate cell substance, which usually contains extracellular matrix proteins, in particular collagen. Connective tissue can occur in particular in the form of loose connective tissue, tight connective tissue, for example in the skin, tendons, ligaments, and the meninges, gelatinous connective tissue and various other types of connective tissue, in particular also supporting tissues, namely cartilage tissue and bone tissue, and they are especially decisive for mobility, the external appearance, the integrity of the internal structures, and the fitness of a body.

The technical problem on which the present invention is based is therefore to provide means and methods which have a biological activity with regard to connective tissue, in particular connective tissue of the skin, tendons, ligaments, meninges, cartilage, and bones, and thus allow, on the one hand, the treatment of related diseases with this connective tissue and, on the other hand, to maintain a condition of such connective tissue that is considered to be desirable.

The present invention is also based on the technical problem of providing synthetic or recombinant collagen peptides which overcome the aforementioned disadvantages, in particular which can be produced recombinantly in a standardized, reliable, and precisely defined form, even on a larger industrial and cost-effective scale, and which, due to their biological efficacy are suitable for use in a method for the therapeutic treatment of the human or animal body.

The present invention solves the technical problem underlying it by providing the teachings of the independent claims, in particular also the teachings of the preferred embodiments in the description and in the dependent claims.

The present invention relates to a synthetic or recombinant collagen peptide, in particular a synthetically or recombinantly produced collagen peptide, for use in a method for the therapeutic treatment of the human or animal body having a molecular weight in a range from 0.18 to 10.0 kDa, in particular 0.18 to 5.0 kDa, in particular 1.1 to 5.0 kDa.

The synthetic or recombinant collagen peptides claimed according to the invention, in particular synthetically or recombinantly produced collagen peptides having a molecular weight in a range from 0.18 to 10.0 kDa, in particular 0.18 to 5.0 kDa, in particular 1.1 to 5.0 kDa, advantageously show a biological activity, in particular in connective tissue, especially in connective tissue of the skin, the intestine, in particular the inner wall of the intestine, blood vessels, the gums, and meninges, connective tissue of the tendons and ligaments and supporting tissue, especially cartilage tissue and bone tissue, and are therefore suitable in particular for use in a method for the therapeutic or non-therapeutic treatment of the human or animal body.

According to a preferred embodiment of the present invention, the synthetic or recombinant collagen peptide, in particular the synthetically or recombinantly produced collagen peptide, has the amino acid sequence SEQ ID No. 1, SEQ ID No. 6 or a function-maintaining sequence modification of one of these amino acid sequences, and the collagen peptide according to the invention preferably consists of one of these amino acid sequences.

The synthetic or recombinant collagen peptide, in particular the synthetically or recombinantly produced collagen peptide, preferably has the amino acid sequence SEQ ID No. 1, and the collagen peptide according to the invention preferably consists of this amino acid sequence.

The synthetic or recombinant collagen peptide, in particular the synthetically or recombinantly produced collagen peptide, preferably has the amino acid sequence SEQ ID No. 6, and the collagen peptide according to the invention preferably consists of this amino acid sequence.

According to a further embodiment of the present invention, the synthetic or recombinant collagen peptide, in particular the synthetically or recombinantly produced collagen peptide, has a function-preserving sequence modification of one of the amino acid sequences SEQ ID No. 1 or SEQ ID No. 6, and the collagen peptide according to the invention preferably consists of a function-maintaining sequence modification of one of these amino acid sequences.

In a preferred embodiment of the present invention, the synthetic or recombinant collagen peptide, in particular the synthetically or recombinantly produced collagen peptide, has an amino acid sequence selected from SEQ ID No. 1 to 5, in particular selected from SEQ ID No. 1 to 4, and the collagen peptide according to the invention preferably consists of one of these amino acid sequences. According to this embodiment, the synthetic or recombinant collagen peptide, in particular the synthetically or recombinantly produced collagen peptide, accordingly does not have any hydroxyproline in its amino acid sequence.

In a preferred embodiment of the present invention, the synthetic or recombinant collagen peptide, in particular the synthetically or recombinantly produced collagen peptide, has an amino acid sequence selected from SEQ ID No. 1 to 5, in particular selected from SEQ ID No. 1 to 4, the collagen peptide according to the invention preferably consists of one of these amino acid sequences, wherein at least one prolyl and/or at least one lysyl residue is hydroxylated. According to this embodiment, the synthetic or recombinant collagen peptide, in particular the synthetically or recombinantly produced collagen peptide, accordingly has at least one hydroxyproline and/or at least one hydroxylysine in its amino acid sequence.

In a further preferred embodiment of the present invention, the synthetic or recombinant collagen peptide, in particular the synthetically or recombinantly produced collagen peptide, has an amino acid sequence selected from SEQ ID No. 6 to 10, in particular selected from SEQ ID No. 6 to 9, and the collagen peptide according to the invention preferably consists of one of these amino acid sequences.

According to a further preferred embodiment, the synthetic or recombinant collagen peptide, in particular the synthetically or recombinantly produced collagen peptide, has an amino acid sequence selected from the group consisting of SEQ ID No. 1 to 10, in particular selected from the group consisting of SEQ ID No. 1 to 4 and 6 to 9.

The synthetic or recombinant collagen peptide, in particular the synthetically or recombinantly produced collagen peptide, preferably consists of one of the amino acid sequences selected from the group consisting of SEQ ID No. 1 to 10, in particular selected from the group consisting of SEQ ID No. 1 to 4 and 6 to 9.

According to a further preferred embodiment of the present invention, the synthetic or recombinant collagen peptide, in particular the synthetically or recombinantly produced collagen peptide, has the amino acid sequence SEQ ID No. 15, SEQ ID No. 21 or a function-maintaining sequence modification of one of these amino acid sequences, and the collagen peptide according to the invention preferably consists of one of these amino acid sequences.

The synthetic or recombinant collagen peptide, in particular the synthetically or recombinantly produced collagen peptide, preferably has the amino acid sequence SEQ ID No. 15, and the collagen peptide according to the invention preferably consists of this amino acid sequence.

The synthetic or recombinant collagen peptide, in particular the synthetically or recombinantly produced collagen peptide, preferably has the amino acid sequence SEQ ID No. 21, and the collagen peptide according to the invention preferably consists of this amino acid sequence.

According to a further embodiment of the present invention, the synthetic or recombinant collagen peptide, in particular the synthetically or recombinantly produced collagen peptide, has a function-preserving sequence modification of one of the amino acid sequences SEQ ID No. 15 or SEQ ID

No. 21, and the collagen peptide according to the invention preferably consists of a function-maintaining sequence modification of one of these amino acid sequences.

In a preferred embodiment of the present invention, the synthetic or recombinant collagen peptide, in particular the synthetically or recombinantly produced collagen peptide, has an amino acid sequence selected from SEQ ID No. 15 and SEQ ID No. 16, and the collagen peptide according to the invention preferably consists of one of these amino acid sequences. According to this embodiment, the synthetic or recombinant collagen peptide, in particular the synthetically or recombinantly produced collagen peptide, accordingly does not have any hydroxyproline in its amino acid sequence.

In a preferred embodiment of the present invention, the synthetic or recombinant collagen peptide, in particular the synthetically or recombinantly produced collagen peptide, has an amino acid sequence selected from SEQ ID No. 15 and SEQ ID No. 16, and the collagen peptide according to the invention preferably consists of one of these amino acid sequences, wherein at least one prolyl and/or at least one lysyl residue is hydroxylated. According to this embodiment, the synthetic or recombinant collagen peptide, in particular the synthetically or recombinantly produced collagen peptide, accordingly has at least one hydroxyproline and/or at least one hydroxylysine in its amino acid sequence.

In a further preferred embodiment of the present invention, the synthetic or recombinant collagen peptide, in particular the synthetically or recombinantly produced collagen peptide, has an amino acid sequence selected from SEQ ID No. 21 and 22, the collagen peptide according to the invention preferably consists of one of these amino acid sequences.

The synthetic or recombinant collagen peptide, in particular the synthetically or recombinantly produced collagen peptide, particularly preferably has the amino acid sequence SEQ ID No. 1, SEQ ID No. 15 or a function-maintaining sequence modification of one of these amino acid sequences, and the collagen peptide according to the invention preferably consists of one of these amino acid sequences.

According to a further preferred embodiment, the synthetic or recombinant collagen peptide, in particular the synthetically or recombinantly produced collagen peptide, has an amino acid sequence selected from the group consisting of SEQ ID No. 1 to 30, in particular selected from the group consisting of SEQ ID No. 1 to 4, 6 to 9, 12 to 14, and 18 to 20.

The synthetic or recombinant collagen peptide, in particular the synthetically or recombinantly produced collagen peptide, preferably consists of one of the amino acid sequences selected from the group consisting of SEQ ID No. 1 to 30, in particular selected from the group consisting of SEQ ID No. 1 to 4, 6 to 9, 12 to 14, and 18 to 20.

In a further preferred embodiment of the present invention, the synthetic or recombinant collagen peptide, in particular the synthetically or recombinantly produced collagen peptide, has an amino acid sequence according to SEQ ID No. 7.

The synthetic or recombinant collagen peptide, in particular the synthetically or recombinantly produced collagen peptide, preferably consists of the amino acid sequence according to SEQ ID No. 7.

In a further preferred embodiment of the present invention, the synthetic or recombinant collagen peptide, in particular the synthetically or recombinantly produced collagen peptide, has an amino acid sequence selected from SEQ ID No. 17 to 19, 21, and 22.

The synthetic or recombinant collagen peptide, in particular the synthetically or recombinantly produced collagen peptide, preferably consists of the amino acid sequence selected from SEQ ID No. 17 to 19, 21 and 22.

In a further preferred embodiment of the present invention, the synthetic or recombinant collagen peptide, in particular the synthetically or recombinantly produced collagen peptide, has an amino acid sequence selected from SEQ ID No. 27 to 30.

The synthetic or recombinant collagen peptide, in particular the synthetically or recombinantly produced collagen peptide, preferably consists of the amino acid sequence selected from SEQ ID No. 27 to 30.

In a particularly preferred embodiment, the synthetic or recombinant collagen peptide, in particular the synthetically or recombinantly produced collagen peptide, according to the present invention is a non-hydroxylated collagen peptide.

In a further preferred embodiment, the synthetic or recombinant collagen peptide, in particular the synthetically or recombinantly produced collagen peptide, according to the present invention is a hydroxylated collagen peptide.

The synthetic or recombinant collagen peptide, in particular the synthetically or recombinantly produced collagen peptide, preferably has hydroxylated proline and/or hydroxylated lysine.

The synthetic or recombinant collagen peptide, in particular the synthetically or recombinantly produced collagen peptide, a non-hydroxylated, partially hydroxylated or completely hydroxylated collagen peptide, is preferred. According to a further particularly preferred embodiment, each proline present in the synthetic or recombinant collagen peptide is hydroxylated.

According to a preferred embodiment of the present invention, the synthetic or recombinant collagen peptide, in particular the synthetically or recombinantly produced collagen peptide, is glycosylated. The synthetic or recombinant collagen peptide, in particular the synthetically or recombinantly produced collagen peptide, is preferably glycosylated on at least one hydroxylated lysine. Each hydroxylated lysine of the synthetic or recombinant collagen peptide, in particular of the synthetically or recombinantly produced collagen peptide, is preferably glycosylated.

In a preferred embodiment, the collagen peptide preferably has an amino acid sequence in collagen of types I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, preferably type I, II or III, preferably type I, preferably type II, preferably type III. The collagen peptide according to the invention preferably includes an amino acid sequence occurring in collagen from vertebrates, in particular from fish, amphibians, reptiles, birds and mammals, in particular in human, bovine, porcine, equine or avian collagen of types I, II or III, preferably type I, preferably type II, preferably type III.

The collagen peptide according to the invention particularly preferably includes an amino acid sequence occurring in human collagen, in particular in human type I collagen, preferably in the al chain of human type I collagen.

The collagen peptide according to the invention particularly preferably includes an amino acid sequence occurring in non-human collagen, in particular in non-human type I collagen, preferably in the amino acid sequence occurring in the al chain of non-human type I collagen, in particular an amino acid sequence occurring in bovine, porcine, equine or avian collagen.

In a preferred embodiment of the present invention, the synthetic or recombinant collagen peptide, in particular the synthetically or recombinantly produced collagen peptide, is collagenase-resistant, in particular resistant to digestion by human collagenases.

According to a further preferred embodiment of the present invention, the synthetic or recombinant collagen peptide, in particular the synthetically or recombinantly produced collagen peptide, is capable of synthesizing extracellular matrix proteins such as collagen, proteoglycan and/or elastin, in particular collagen, in connective tissue cells, in particular in osteoblasts, chondrocytes and/or fibroblasts.

In a preferred embodiment of the present invention, the collagen peptide according to the invention is a synthetically produced collagen peptide, preferably a collagen peptide produced by chemical synthesis, in particular solid phase synthesis, preferably Merrifield synthesis, Bailey peptide synthesis or chemoenzymatic synthesis. In a further preferred embodiment of the present invention, the collagen peptide according to the invention is a recombinantly produced collagen peptide.

In a preferred embodiment of the present invention, the synthetic or recombinant collagen peptide, in particular the synthetically or recombinantly produced collagen peptide, has no amino acid modification, in particular no hydroxylation. The synthetic or recombinant collagen peptide, in particular the synthetically or recombinantly produced collagen peptide, particularly preferably has no hydroxylated and/or glycosylated amino acids.

According to a preferred embodiment of the present invention, the synthetic or recombinant collagen peptide, in particular the synthetically or recombinantly produced collagen peptide for use in a method for the therapeutic treatment of the human or animal body, has a molecular weight in a range from 0.18 to 10.0 kDa, preferably 0.18 to 8.0 kDa, preferably 0.18 to 6.0 kDa, preferably 0.18 to 5.0 kDa, preferably 0.2 to 5.0 kDa, preferably 0.3 to 5.0 kDa, preferably 0.4 to 5.0 kDa, preferably 0.5 to 5.0 kDa, preferably 0.6 to 5.0 kDa, preferably 0.7 to 5.0 kDa, preferably 0.8 to 5.0 kDa, preferably 0.9 to 5.0 kDa, preferably 1.0 to 5.0 kDa, preferably 1.1 to 5.0 kDa, preferably 1.2 to 5.0 kDa, preferably 1.3 to 5.0 kDa, preferably 1.4 to 5.0 kDa, preferably 1.5 to 5.0 kDa, preferably 1.6 to 5.0 kDa, preferably 1.7 to 5.0 kDa, preferably 1.8 to 5.0 kDa, preferably 1.9 to 5.0 kDa, preferably 2.0 to 5.0 kDa, preferably 2.1 to 4.9 kDa, preferably 2.2 to 4.8 kDa, preferably 2.3 to 4.7 kDa, preferably 2.4 to 4.6 kDa, preferably 2.5 to 4.5 kDa, preferably 1.2 to 3.2, preferably 1.3 to 3.0 kDa, preferably 1.5 to 3.0 kDa, preferably 1.8 to 3.0 kDa, preferably 2.0 to 3.0 kDa.

According to a further preferred embodiment of the present invention, the synthetic or recombinant collagen peptide, in particular the synthetically or recombinantly produced collagen peptide for use in a method for the therapeutic treatment of the human or animal body has a molecular weight in a range from 0.18 to 0.98, preferably 0.19 to 0.98 kDa, preferably 0.20 to 0.98 kDa, preferably 0.25 to 0.98 kDa, preferably 0.30 to 0.98 kDa, preferably 0.35 to 0.98 kDa, preferably 0.40 to 0.98 kDa, preferably 0.45 to 0.98 kDa, preferably 0.50 to 0.98 kDa.

The present invention also relates in a preferred embodiment to the synthetic or recombinant collagen peptide according to the invention, in particular the synthetically or recombinantly produced collagen peptide, to be used in a method for preventing and/or treating bone diseases, in particular osteoporosis.

In a preferred embodiment the present invention relates to the synthetic or recombinant collagen peptide according to the invention, in particular the synthetically or recombinantly produced collagen peptide, to be used in a method for preventing and/or treating sarcopenia.

In a preferred embodiment the present invention relates to the synthetic or recombinant collagen peptide according to the invention, in particular the synthetically or recombinantly produced collagen peptide, to be used in a method for preventing and/or treating degenerative loss of muscle mass.

In a preferred embodiment the present invention relates to the synthetic or recombinant collagen peptide according to the invention, in particular the synthetically or recombinantly produced collagen peptide, to be used in a method for preventing and/or treating cartilage disease, especially osteoarthritis or arthritis.

In a preferred embodiment the present invention relates to the synthetic or recombinant collagen peptide according to the invention, in particular the synthetically or recombinantly produced collagen peptide, to be used in a method for improving muscle strength.

In a preferred embodiment the present invention relates to the synthetic or recombinant collagen peptide according to the invention, in particular the synthetically or recombinantly produced collagen peptide, to be used in a method for preventing and/or treating a pathological condition characterized by reduced mitochondrial activity, in particular for preventing and/or treating a pathological condition characterized by reduced endurance.

In a preferred embodiment the present invention relates to the synthetic or recombinant collagen peptide according to the invention, in particular the synthetically or recombinantly produced collagen peptide, to be used in a method for stimulation of fat loss.

In a preferred embodiment the present invention relates to the synthetic or recombinant collagen peptide according to the invention, in particular the synthetically or recombinantly produced collagen peptide, to be used in a method for reduction in body weight.

In a preferred embodiment the present invention relates to the synthetic or recombinant collagen peptide according to the invention, in particular the synthetically or recombinantly produced collagen peptide, to be used in a method for preventing and/or treating degenerative joint diseases, in particular osteoarthritis, rheumatoid arthritis, rheumatic diseases, spondylitis and/or fibromyalgia.

In a preferred embodiment the present invention relates to the synthetic or recombinant collagen peptide according to the invention, in particular the synthetically or recombinantly produced collagen peptide, to be used in a method for preventing and/or treating diseases of the tendons or ligaments.

In a preferred embodiment the present invention relates to the synthetic or recombinant collagen peptide according to the invention, in particular the synthetically or recombinantly produced collagen peptide, to be used in a method for preventing and/or treating skin diseases, especially psoriasis vulgaris, acne, atopic dermatitis, chronic pruritus and/or rosacea.

In a preferred embodiment the present invention relates to the synthetic or recombinant collagen peptide according to the invention, in particular the synthetically or recombinantly produced collagen peptide, to be used in a method for treating wounds, in particular chronic wounds, acute wounds and/or burns.

In a preferred embodiment the present invention relates to the synthetic or recombinant collagen peptide according to the invention, in particular the synthetically or recombinantly produced collagen peptide, to be used in a method for preventing and/or treating degenerative nerve diseases.

In a preferred embodiment the present invention relates to the synthetic or recombinant collagen peptide according to the invention, in particular the synthetically or recombinantly produced collagen peptide, to be used in a method for preventing and/or treating dementia.

In a preferred embodiment the present invention relates to the synthetic or recombinant collagen peptide according to the invention, in particular the synthetically or recombinantly produced collagen peptide, to be used in a method for preventing and/or treating Alzheimer's.

In a preferred embodiment, the present invention relates to the synthetic or recombinant collagen peptide according to the invention, in particular the synthetically or recombinantly produced collagen peptide, to be used in a method for preventing and/or treating a pathological condition characterized by a reduction in mental performance.

In a preferred embodiment the present invention relates to the synthetic or recombinant collagen peptide according to the invention, in particular the synthetically or recombinantly produced collagen peptide, to be used in a method for preventing and/or treating diseases related to malfunctions of the blood-brain barrier, in particular the structure and/or function of the meninges.

In a preferred embodiment the present invention relates to the synthetic or recombinant collagen peptide according to the invention, in particular the synthetically or recombinantly produced collagen peptide, to be used in a method for preventing and/or treating intestinal diseases, particularly inflammatory bowel disease.

In a preferred embodiment the present invention relates to the synthetic or recombinant collagen peptide according to the invention, in particular the synthetically or recombinantly produced collagen peptide, to be used in a method for preventing and/or treating diseases of the cardiovascular system, in particular the structure and/or function of the blood vessels, in particular the vascular wall, in particular for preventing and/or treating high blood pressure and/or circulatory disorders.

In a preferred embodiment the present invention relates to the synthetic or recombinant collagen peptide according to the invention, in particular the synthetically or recombinantly produced collagen peptide, to be used in a method for preventing and/or treating periodontal diseases.

In one preferred embodiment, the present invention also relates to the non-therapeutic use of the collagen peptide according to the invention for the visual and structural improvement of the skin, in particular for reducing wrinkles, improving skin elasticity, increasing skin resilience, increasing the moisture content of the skin, reducing cellulite and/or reducing stretch marks, especially stretch marks from pregnancy.

In a further preferred embodiment, the present invention relates to the non-therapeutic use of the collagen peptide according to the invention for accelerating the growth of nails and/or reducing the fragility of nails.

In a preferred embodiment, the present invention also relates to the non-therapeutic use of the collagen peptide according to the invention for the visual and structural improvement of the hair, in particular for improving the hair quality, reducing split ends and/or reducing/delaying hair loss.

In a further preferred embodiment, the present invention relates to the non-therapeutic use of the collagen peptide according to the invention for increasing the number of mitochondria and/or mitochondrial activity.

In a further preferred embodiment, the present invention relates to the non-therapeutic use of the collagen peptide according to the invention for improving endurance performance.

In a further preferred embodiment, the present invention relates to the non-therapeutic use of the collagen peptide according to the invention for improving mental performance.

In a preferred embodiment of the present invention, the synthetic or recombinant collagen peptide according to the invention is used alone, i.e., without further substances, for use in one of the applications provided according to the invention.

In a further embodiment of the present invention, the synthetic or recombinant collagen peptide according to the invention is used as the sole agent exhibiting biological activity in an application provided according to the invention.

In a further preferred embodiment, the collagen peptide according to the invention is used together with at least one further agent, in particular a further biologically active agent, in an application provided according to the invention.

The present invention also relates to a pharmaceutical composition comprising a collagen peptide according to the invention and at least one pharmaceutically acceptable additive, as well as the pharmaceutical composition for use in a method for the therapeutic treatment of the human or animal body, in particular for use in at least one of the aforementioned indications. Accordingly, it can be provided that the collagen peptide according to the invention is administered in the form of a pharmaceutical preparation. The pharmaceutical preparation according to the invention is administered particularly advantageously, for example, in the form of tablets, lozenges, chewable tablets, capsules, bite capsules, coated tablets, pastilles, juices, gels or ointments.

The present invention also relates to a food supplement comprising a collagen peptide according to the invention and at least one food-acceptable additive, as well as the food supplement for use in a method for the therapeutic treatment of the human or animal body, in particular for use in at least one of the aforementioned indications. Accordingly, it can be provided that the collagen peptide according to the invention is administered in the form of a food supplement. The food supplement according to the invention is particularly advantageously in the form of a tablet, coated tablet, pastille, solution, suspension or gel, for example, in an ampoule, as granules or powder. Due to its good solubility, the collagen peptide according to the invention may also be added to various beverages without causing cloudiness.

The present invention also relates to a cosmetic product comprising a collagen peptide according to the invention and at least one skin-compatible additive, as well as the cosmetic product for use in a method for the therapeutic treatment of the human or animal body, in particular for use in at least one of the aforementioned indications. Accordingly, it can be provided that the collagen peptide according to the invention is administered in the form of a cosmetic composition. The cosmetic composition according to the invention is particularly advantageously administered, for example, in the form of lotions, ointments, creams, gels, powders, syringes or sprays.

The invention also relates to a food or luxury food item comprising a collagen peptide according to the invention, as well as the food or luxury food item for use in a method for the therapeutic treatment of the human or animal body, in particular for use in at least one of the aforementioned indications. According to a preferred embodiment, the food or luxury food item is a chocolate bar, protein bar, cereal bar, instant powder for the preparation of beverages, milk, milk products, for example yogurt, whey or quark and milk substitutes, for example soy milk, rice milk, almond milk, and coconut milk (so-called functional food) or a drink, e.g. soft drink or fitness drink.

If the collagen peptide according to one preferred embodiment of the invention is not used as the sole physiologically active component of a product, in particular of a pharmaceutical preparation, of a food supplement, of a cosmetic preparation or of a food or luxury food item, it may be combined with one or more other components that have a positive effect on general health, in particular on endurance performance. Such components are preferably selected from the group consisting of vitamin C, vitamins of the B, D, E and K series, omega-3 fatty acids, omega-6 fatty acids, conjugated linolenic acids, caffeine and its derivatives, guarana extract, rose hip extract, green tea extract, epigallocatechin gallate, creatine, L-carnitine, α-lipoic acid, N-acetylcysteine, NADH, D-ribose, magnesium aspartate, antioxidants such as anthocyanins, carotenoids, flavonoids, resveratrol, glutathione and superoxide dismutase (SOD), cannabidinoids such as cannabidiol (CBD), adaptogens such as Rhodiola rosea, Panax ginseng, Withania somnifera, shiitake, Ganoderma lucidum Lepidium meyenii, minerals such as iron, magnesium, calcium, zinc, selenium and phosphorus, as well as other proteins, hydrolysates and peptides such as soy, wheat, and whey protein.

In a further preferred embodiment of the present invention, the products according to the invention, in particular the pharmaceutical preparation, the dietary supplement, the cosmetic composition or the food or luxury food item, contain no further proteins or peptides in addition to the collagen peptide according to the invention, in particular no further collagen peptides.

In one preferred embodiment of the invention, the collagen peptide is administered in an amount of 1 to 40 g per day, preferably from 1 to 30 g per day, preferably from 1 to 20 g per day, preferably from 1 to 15 g per day, preferably from 2.5 to 30 g per day, preferably 2.5 to 20 g per day, preferably 2.5 to 15 g per day, preferably 2.5 to 10 g per day, preferably 4 to 15 g per day, preferably 4 to 12 g per day, more preferably from 5 to 25 g per day, preferably from 5 to 15 g per day, preferably from 10 to 25 g per day, preferably from 12 to 22 g per day, preferably 6 to 15 g per day, in particular from 2.5 to 7.5 g per day, preferably 2.5 to 5 g per day.

The present invention also relates to methods for preventing and/or treating the aforementioned indications, in particular of the aforementioned therapeutic indications, according to which a sufficient amount of the recombinantly or synthetically produced collagen peptide according to the invention is administered to the human or animal body for the therapeutic purpose, optionally with an additive.

The present invention also relates to a non-therapeutic method for improving muscle strength, for increasing muscle mass, for stimulating fat loss, for reducing body weight, for maintaining and/or improving bone health, for maintaining and/or improving skin health, for maintaining and/or improving the intestinal health, for maintaining and/or improving the blood vessel structure, for maintaining and/or improving the health of the cardiovascular system, for maintaining and/or improving the gums, for maintaining and/or improving the health of the nails and hair of a human or animal body, for maintaining and/or increasing the number of mitochondria and/or mitochondrial activity, for maintaining and/or improving endurance performance or for maintaining and/or improving mental performance, wherein at least one collagen peptide according to the invention is administered to the human or animal body.

In addition, the present invention relates to a method for producing a collagen peptide according to the invention, comprising the method steps:
a) Providing an expression system that has at least one expression cassette, wherein the expression cassette has at least one nucleotide sequence that encodes a collagen peptide having a molecular weight in a range from 0.18 to 10 kDa, in particular 0.18 to 5.0 kDa, in particular 1.1 to 5.0 kDa,
b) Cultivating the expression system under conditions that allow expression of the collagen peptide, and
c) Obtaining the collagen peptide according to the invention.

The method provided according to the invention for producing a collagen peptide according to the invention, in particular a synthetic or recombinant collagen peptide for the therapeutic treatment of the human or animal body having a molecular weight in a range from 0.18 to 10.0 kDa, in particular 0.18 to 5.0 kDa, in particular 1.1 to 5.0 kDa, is characterized in particular by the fact that a precisely defined, recombinantly produced collagen peptide having a molecular weight in a range from 0.18 to 10.0 kDa, in particular 0.18 to 5.0 kDa, in particular 1.1 to 5.0 kDa is obtained, which is suitable, in particular because of its biological effectiveness, for use in a method for the therapeutic treatment of the human or animal body.

The collagen peptide provided according to the invention has a particularly high purity due to its recombinant production method compared to collagen peptides obtained hydrolytically from natural sources. It can be provided in a wide variety of expression systems on an industrial scale without undesired contamination, wherein the collagen peptide according to the invention at the same time advantageously has a biological effectiveness, in particular with regard to uses in a method for the therapeutic treatment of the human or animal body.

The biological activity of the collagen peptides according to the invention found according to the invention and, associated with this, their suitability for use in a method for the therapeutic treatment of the human or animal body, advantageously already applies to the collagen peptides obtained directly from the method according to the invention, without the need for further processing steps. Both the hydroxylated and the non-hydroxylated collagen peptides according to the present invention show a biological activity, in particular at least the same biological activity as collagen peptides obtained from natural sources of the same molecular weight and/or mixtures of collagen peptides obtained from natural sources with a comparable average molecular weight. It is particularly advantageous that the collagen peptides according to the invention surprisingly have a biological activity even in non-hydroxylated form, preferably the same biological activity as hydroxylated collagen peptides obtained from natural sources of the same molecular weight and/or mixtures of hydroxylated collagen peptides obtained from natural sources with a comparable average molecular weight, and particularly preferably have a better biological activity than hydroxylated collagen peptides obtained from natural sources of the same molecular weight and/or mixtures of hydroxylated collagen peptides obtained from natural sources with a comparable average molecular weight.

The hydroxylated and non-hydroxylated collagen peptides according to the present invention preferably show in osteoblasts, fibroblasts and/or chondrocytes, preferably in osteoblasts, preferably in fibroblasts, preferably in chondrocytes, preferably in osteoblasts and fibroblasts, preferably in osteoblasts and chondrocytes, preferably in fibroblasts and chondrocytes, a biological activity, preferably at least the same biological activity as collagen peptides obtained from natural sources of the same molecular weight and/or mixtures of collagen peptides obtained from natural sources with a comparable average molecular weight, particularly preferably better biological activity than collagen peptides obtained from natural sources of the same molecular weight and/or mixtures of collagen peptides obtained from natural sources with comparable average molecular weight.

The biological activity found according to the invention and the synthetic or recombinant collagen peptides, in particular the synthetically or recombinantly produced collagen peptides, of the present invention, in particular in connective tissue, in particular in connective tissue of the skin, the intestine, the blood vessels and the gums, meninges, tendons, ligaments, cartilage and/or bones can be determined in particular using the in vitro tests shown in Examples 3 to 6 for stimulating the synthesis of extracellular matrix proteins in osteoblasts, fibroblasts, and chondrocytes.

In a preferred embodiment, the synthetic or recombinant collagen peptides according to the invention, in particular the synthetically or recombinantly produced collagen peptides, in particular the synthetically or recombinantly produced hydroxylated collagen peptides, in particular the synthetically or recombinantly produced non-hydroxylated collagen peptides, according to the present invention, in particular have a biological activity in at least one, preferably in at least two, preferably in all, of the in vitro tests shown in Examples 3 to 6 for stimulating the synthesis of extracellular matrix proteins in osteoblasts, fibroblasts, and chondrocytes.

The synthetic or recombinant collagen peptides according to the invention, in particular the synthetically or recombinantly produced collagen peptides, in particular the synthetically or recombinantly produced hydroxylated collagen peptides, in particular the synthetically or recombinantly produced non-hydroxylated collagen peptides, of the present invention preferably have the same biological activity as collagen peptides isolated from natural sources of the same molecular weight, and in particular they have the same biological activity in at least one, preferably in at least two, preferably in all of the in vitro tests shown in Examples 3 to 6 for stimulating the synthesis of extracellular matrix proteins in osteoblasts, fibroblasts, and chondrocytes on collagen peptides isolated from natural sources of the same molecular weight.

Particularly preferably, the synthetic or recombinant collagen peptides according to the invention, in particular the synthetically or recombinantly produced collagen peptides, in particular the synthetically or recombinantly produced hydroxylated collagen peptides, in particular the synthetically or recombinantly produced non-hydroxylated collagen peptides, of the present invention have the same biological activity as mixtures of collagen peptides isolated from natural sources, in particular such as mixtures of collagen peptides isolated from natural sources with a comparable average molecular weight, and in particular they exhibit in at least one, preferably in at least two, preferably in all, of the in vitro tests shown in Examples 3 to 6 for the stimulation of the synthesis of extracellular matrix proteins in osteoblasts, fibroblasts, and chondrocytes, the same biological activity as mixtures of collagen peptides isolated from natural sources, in particular such as mixtures of collagen peptides isolated from natural sources with comparable average molecular weights.

The synthetic or recombinant collagen peptides according to the invention, in particular the synthetically or recombinantly produced collagen peptides, in particular the synthetically or recombinantly produced hydroxylated collagen peptides, in particular the synthetically or recombinantly produced non-hydroxylated collagen peptides, of the present invention particularly preferably have a better biological activity than collagen peptides isolated from natural sources of the same molecular weight, and in particular they have a better biological activity in at least one, preferably in at least two, preferably in all of the in vitro tests shown in Examples 3 to 6 for the stimulation of the synthesis of extracellular matrix proteins in osteoblasts, fibroblasts, and chondrocytes as collagen peptides of the same molecular weight isolated from natural sources.

The synthetic or recombinant collagen peptides according to the invention, in particular the synthetically or recombinantly produced collagen peptides, in particular the synthetically or recombinantly produced hydroxylated collagen peptides, in particular the synthetically or recombinantly produced non-hydroxylated collagen peptides, of the present invention particularly preferably have a better biological activity than mixtures of collagen peptides from natural sources, in particular such as mixtures of collagen peptides isolated from natural sources with a comparable average molecular weight, and in particular have in at least one, preferably in at least two, preferably in all, of the in vitro tests shown in Examples 3 to 6 for the stimulation of the synthesis of extracellular matrix proteins in osteoblasts, fibroblasts and chondrocytes, a better biological activity than mixtures of collagen peptides isolated from natural sources, in particular such as mixtures of collagen peptides isolated from natural sources with a comparable average molecular weight.

The expression system provided in step a), is preferably a host cell, in particular a prokaryotic or eukaryotic cell.

The expression system is preferably a host cell selected from the group made up of bacterial cells, yeast cells, fungal cells, mammalian cells, insect cells, and plant cells.

The expression system, in particular the host cell, is preferably a bacterial cell, in particular, of the species Escherichia coli or Bacillus subtilis.

In a further preferred embodiment, the expression system, in particular the host cell, is a yeast cell, in particular of the species Saccharomyces cerevisiae, Pichia pastoris or Ogataea angusta (Hansenula polymorpha).

The expression system, in particular the host cell, is preferably a fungal cell, in particular of the Aspergillus niger species.

In a further preferred embodiment of the present invention, the expression system, in particular the host cell, is a mammalian cell, in particular a CHO cell, a HeLa cell or a HEK293 cell.

The expression system, in particular the host cell, is preferably an insect cell, in particular an Sf-9, Sf-21 or Tn-5 cell.

The expression system, in particular the host cell, is preferably a plant cell, in particular a maize or tobacco cell.

In a further preferred embodiment of the present invention, the expression system provided in step a) is a host cell which is capable of hydroxylating proline residues, lysine residues or proline and lysine residues of the expressed collagen peptide. The expression system provided in step a) is preferably a host cell which is capable of hydroxylating proline residues, lysine residues or proline and lysine residues of the expressed collagen peptide.

The expression system provided in step a) is preferably an expression system which has prolyl hydroxylase activity and/or lysyl hydroxylase activity. The expression system provided in step a) is preferably a host cell which has prolyl hydroxylase activity and/or lysyl hydroxylase activity.

In one preferred embodiment, the expression system provided in step a) is a host cell including at least one expression cassette which comprises a prolyl 4-hydroxylase-encoding polynucleotide sequence. The expression system provided in step a) is particularly preferably a host cell including at least one expression cassette which comprises a prolyl 4-hydroxylase-encoding polynucleotide sequence, so that an in vivo hydroxylated collagen peptide preparation is obtained in method step c).

In one preferred embodiment, the expression system provided in step a) is a host cell including at least one expression cassette which comprises a lysyl hydroxylase-encoding polynucleotide sequence. The expression system provided in step a) is particularly preferably a host cell including at least one expression cassette which comprises a prolyl 4-hydroxylase-encoding polynucleotide sequence, so that an in vivo hydroxylated collagen peptide preparation is obtained in method step c).

In a further preferred embodiment of the present invention, the expression system provided in step a) is a host cell including at least one expression cassette which comprises a prolyl-4-hydroxylase-encoding polynucleotide sequence and at least one expression cassette which comprises a lysyl hydroxylase-encoding polynucleotide sequence. The expression system provided in step a) is particularly preferably a host cell including at least one expression cassette which comprises a prolyl 4-hydroxylase-encoding polynucleotide sequence and at least one expression cassette which comprises a lysyl hydroxylase-encoding polynucleotide sequence, so that an in vivo hydroxylated collagen peptide preparation is obtained in method step c).

Accordingly, the present invention also comprises a method for producing a collagen peptide according to the invention, in particular, an in vivo hydroxylated collagen peptide, comprising the method steps
a) Providing an expression system including at least one expression cassette, wherein the expression cassette includes at least one nucleotide sequence that encodes a collagen peptide having a molecular weight in a range of 0.18 to 10.0 kDa, in particular 0.18 to 5.0 kDa, in particular 1.1 to 5.0 kDa, and wherein the expression system is not capable of hydroxylating proline residues, lysine residues or proline and lysine residues of the expressed collagen peptide,
b) Cultivating the expression system under conditions which enable the expression and hydroxylation of the collagen peptide,
c) Obtaining the collagen peptide according to the invention, in particular the in vivo hydroxylated collagen peptide,

With the aid of the aforementioned method, it is thus advantageously possible to produce an in vivo hydroxylated recombinantly produced collagen peptide having a specific molecular weight in a range from 0.18 to 10.0 kDa, in particular 0.18 to 5.0 kDa, in particular 1.1 to 5.0 kDa, which, depending on the cell-based expression system used, is characterized by a specific pattern of post-translational modifications, in particular hydroxylations and glycosylations. In this way, it is advantageously particularly possible, directly, i.e. without the need for subsequent modification, to obtain a collagen peptide with the desired biological activity, in particular a recombinantly produced collagen peptide for use in a method for the therapeutic treatment of the human or animal body.

In a preferred embodiment, the recombinant in vivo hydroxylated collagen peptide produced according to the invention, exhibits a biological activity in at least one, preferably in at least two, preferably in all of the in vitro tests shown in Examples 3 to 6, for stimulating the synthesis of extracellular matrix proteins in osteoblasts, fibroblasts and chondrocytes.

The recombinant in vivo hydroxylated collagen peptide produced according to the invention preferably has the same biological activity as collagen peptides isolated from natural sources of the same molecular weight, in particular it has in at least one, preferably in at least two, preferably in all, of the in vitro tests shown in Examples 3 to 6 for stimulating the synthesis of extracellular matrix proteins in osteoblasts, fibroblasts and chondrocytes, the same biological activity as collagen peptides isolated from natural sources of the same molecular weight.

The recombinant in vivo hydroxylated collagen peptide produced according to the invention particularly preferably has the same biological activity as mixtures of collagen peptides isolated from natural sources, in particular as mixtures of collagen peptides isolated from natural sources with a comparable average molecular weight, and in particular it has in at least one, preferably at least two, preferably in all of the in vitro tests shown in Examples 3 to 6 for stimulating the synthesis of extracellular matrix proteins in osteoblasts, fibroblasts and chondrocytes, the same biological activity as mixtures of collagen peptides isolated from natural sources, in particular as mixtures of collagen peptides isolated from natural sources with comparable average molecular weight.

The recombinant in vivo hydroxylated collagen peptide produced according to the invention particularly preferably has a better biological activity than collagen peptides isolated from natural sources of the same molecular weight, in particular it has in at least one, preferably in at least two, preferably in all of the in vitro tests shown in Examples 3 to 6 for stimulating the synthesis of extracellular matrix proteins in osteoblasts, fibroblasts and chondrocytes, a better biological activity than collagen peptides isolated from natural sources of the same molecular weight.

The recombinant in vivo hydroxylated collagen peptide produced according to the invention particularly preferably has a better biological activity than mixtures of collagen peptides isolated from natural sources, in particular such as mixtures of collagen peptides isolated from natural sources with a comparable average molecular weight, in particular it has in at least one, preferably in at least two, preferably in all of the in vitro tests shown in Examples 3 to 6 for stimulating the synthesis of extracellular matrix proteins in osteoblasts, fibroblasts and chondrocytes, a better biological activity than mixtures of collagen peptides isolated from natural sources, in particular than mixtures of collagen peptides isolated from natural sources with comparable average molecular weight.

According to a further embodiment of the present invention, the expression system provided in step a) is an expression system which is not capable of bringing about a hydroxylation of proline residues, lysine residues or proline and lysine residues of the expressed collagen peptide, in particular, the expression system provided in step a) does not exhibit prolyl hydroxylase activity and lysyl hydroxylase activity.

The present invention thus comprises a method for producing a collagen peptide according to the invention, in particular a non-hydroxylated collagen peptide, comprising the method steps
a) Providing an expression system including at least one expression cassette, wherein the expression cassette includes at least one nucleotide sequence that encodes a collagen peptide having a molecular weight in a range of 0.18 to 10.0 kDa, in particular 0.18 to 5.0 kDa, in particular 1.1 to 5.0 kDa, and wherein the expression system is not capable of hydroxylating proline residues, lysine residues or proline and lysine residues of the expressed collagen peptide,
b) Cultivating the expression system under conditions which enable the expression of the collagen peptide.
c) Obtaining the collagen peptide according to the invention, especially the non-hydroxylated collagen peptide.

In a preferred embodiment, the recombinant non-hydroxylated collagen peptide produced according to the invention exhibits a biological activity, in particular in at least one, preferably it exhibits biological activity in at least two, preferably in all of the in vitro tests shown in Examples 3 to 6, for stimulating the synthesis of extracellular matrix proteins in osteoblasts, fibroblasts and chondrocytes.

The recombinant non-hydroxylated collagen peptide produced according to the invention preferably has the same biological activity as collagen peptides isolated from natural sources of the same molecular weight, in particular it has in at least one, preferably in at least two, preferably in all in vitro tests shown in Examples 3 to 6 for stimulating the synthesis of extracellular matrix proteins in osteoblasts, fibroblasts, and chondrocytes, the same biological activity as collagen peptides isolated from natural sources of the same molecular weight.

The recombinant non-hydroxylated collagen peptide produced according to the invention particularly preferably has the same biological activity as mixtures of collagen peptides isolated from natural sources, in particular as mixtures of collagen peptides isolated from natural sources with a comparable average molecular weight, and in particular it has in at least one, preferably in at least two, preferably in all of the in vitro tests shown in Examples 3 to 6 for stimulating the synthesis of extracellular matrix proteins in osteoblasts, fibroblasts and chondrocytes, the same biological activity as mixtures of collagen peptides isolated from natural sources, in particular as mixtures of collagen peptides isolated from natural sources with comparable average molecular weight.

The recombinant non-hydroxylated collagen peptide produced according to the invention particularly preferably has better biological activity than collagen peptides isolated from natural sources of the same molecular weight, and in particular it has in at least one, preferably in at least two, preferably in all vitro tests shown in examples 3 to 6 for stimulating the synthesis of extracellular matrix proteins in osteoblasts, fibroblasts, and chondrocytes, a better biological activity than collagen peptides isolated from natural sources of the same molecular weight.

The recombinant non-hydroxylated collagen peptide produced according to the invention particularly preferably has a better biological activity than mixtures of collagen peptides isolated from natural sources, in particular such as mixtures of collagen peptides isolated from natural sources with a comparable average molecular weight, and in particular it has in at least one, preferably in at least two, and preferably in all of the in vitro tests shown in Examples 3 to 6 for stimulating the synthesis of extracellular matrix proteins in osteoblasts, fibroblasts and chondrocytes, a better biological activity than mixtures of collagen peptides isolated from natural sources, in particular than mixtures of collagen peptides isolated from natural sources with comparable average molecular weight.

According to one preferred embodiment of the present invention, the at least one nucleotide sequence of the at least one expression cassette is codon-optimized, i.e., those codons in the nucleotide sequence that are not used or are not preferably used by the translation system of the provided expression system, in particular by the provided cell-based expression system, in particular by the provided host cell, are replaced by those which are preferably used by the translation system of the provided expression system, in particular by the provided cell-based expression system, in particular by the provided host cell, without thereby changing the amino acid sequence of the encoded peptide or protein.

In one preferred embodiment of the present invention, the collagen peptide encoded by the nucleotide sequence is a collagen peptide from a vertebrate, in particular a mammal, for example a human or a non-human mammal, for example a horse, donkey, kangaroo, sheep, rodent, pig or cattle, a bird, for example a chicken, a fish, an amphibian, a reptile or an invertebrate, for example, a jellyfish.

The collagen peptide encoded by the nucleotide sequence is preferably selected from types I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, preferably type I, preferably type II, preferably type III.

The collagen peptide encoded by the nucleotide sequence is preferably a naturally occurring collagen peptide. In a further preferred embodiment of the present invention, the collagen peptide encoded by the nucleotide sequence is not a naturally occurring collagen peptide. The collagen peptide encoded by the nucleotide sequence is preferably a genetically modified collagen peptide.

According to a preferred embodiment of the present invention, the at least one nucleotide sequence encodes a collagen peptide having a molecular weight in a range of preferably 0.18 to 10.0 kDa, preferably 0.18 to 8.0 kDa, preferably 0.18 to 6.0 kDa, preferably 0.18 to 5.0 kDa, preferably 0.2 to 5.0 kDa, preferably 0.3 to 5.0 kDa, preferably 0.4 to 5.0 kDa, preferably 0.5 to 5.0 kDa, preferably 0.6 to 5.0 kDa, preferably 0.7 to 5.0 kDa, preferably 0.8 to 5.0 kDa, preferably 0.9 to 5.0 kDa, preferably 1.0 to 5.0 kDa, preferably 1.1 to 5.0 kDa, preferably 1.2 to 5.0 kDa, preferably 1.3 to 5.0 kDa, preferably 1.4 to 5.0 kDa, preferably 1.5 to 5.0 kDa, preferably 1.6 to 5.0 kDa, preferably 1.7 to 5.0 kDa, preferably 1.8 to 5.0 kDa, preferably 1.9 to 5.0 kDa, preferably 2.0 to 5.0 kDa, preferably 2.1 to 4.9 kDa, preferably 2.2 to 4.8 kDa, preferably 2.3 to 4.7 kDa, preferably 2.4 to 4.6 kDa, preferably 2.5 to 4.5 kDa, preferably 1.2 to 3.2, preferably 1.3 to 3.0 kDa, preferably 1.5 to 3.0 kDa, preferably 1.8 to 3.0 kDa, preferably 2.0 to 3.0 kDa.

According to a further preferred embodiment of the present invention, the at least one nucleotide sequence encodes a collagen peptide having a molecular weight in a range from 0.18 to 0.98, preferably 0.19 to 0.98 kDa, preferably 0.20 to 0.98 kDa, preferably 0.25 to 0.98 kDa, preferably 0.30 to 0.98 kDa, preferably 0.35 to 0.98 kDa, preferably 0.40 to 0.98 kDa, preferably 0.45 to 0.98 kDa, preferably 0.50 to 0.98 kDa.

In a preferred embodiment of the present invention, the collagen peptide produced with one of the aforementioned methods according to the invention, in particular the synthetic or recombinant collagen peptide for use in a method for the therapeutic treatment of the human or animal body having a molecular weight in a range from 0.18 to 10.0 kDa, in particular 0.18 to 5.0 kDa, in particular 1.1 to 5.0 kDa, is a non-hydroxylated, partially hydroxylated or fully hydroxylated collagen peptide, preferably a non-hydroxylated collagen peptide, preferably a partially hydroxylated collagen peptide, preferably a fully hydroxylated collagen peptide.

In a preferred embodiment of the present invention, the collagen peptide produced using one of the methods according to the invention, in particular the synthetic or recombinant collagen peptide for use in a method for the therapeutic treatment of the human or animal body, having a molecular weight in a range from 0.18 to 10.0 kDa, in particular 0.18 to 5.0 kDa, in particular 1.1 to 5.0 kDa, is a glycosylated collagen peptide. The collagen peptides are preferably glycosylated in vivo, preferably glycosylated ex vivo.

In a further preferred embodiment of the present invention, the collagen peptide produced using one of the methods according to the invention is a non-glycosylated collagen peptide.

In a particularly preferred embodiment of the present invention, in particular one of the methods for producing a collagen peptide according to the invention, the at least one expression cassette of the expression system has several, preferably at least two, preferably at least three, preferably at least four, preferably at least five, nucleotide sequences strung together that each encode a collagen peptide having a molecular weight in a range from 0.18 to 10.0 kDa, in particular 0.18 to 5.0 kDa, in particular 1.1 to 5.0 kDa.

According to this embodiment, a precursor collagen peptide is first synthesized by the expression system, from which collagen peptides according to the invention can then be obtained by cleavage, in particular enzymatic cleavage.

The size of the collagen precursor peptide is preferably 2 to 100 kDa, preferably 3 to 80 kDa, preferably 4 to 60 kDa, preferably 5 to 50 kDa, preferably 10 to 45 kDa. In a preferred embodiment, the precursor collagen peptide comprises 2 to 40 collagen peptides according to the invention, preferably 3 to 30 collagen peptides according to the invention, preferably 5 to 20 collagen peptides according to the invention.

In a preferred embodiment, the multiple, preferably at least two, preferably at least three, preferably at least four, preferably at least five, are each a collagen peptide with a molecular weight in a range from 0.18 to 10.0 kDa, in particular 0.18 to 5.0 kDa, in particular 1.1 to 5.0 kDa encoding, strung-together nucleotide sequences separated by sequences that enable cleavage, in particular enzymatic cleavage, of the collagen peptide expressed in step b) into several, preferably at least two, preferably at least three, preferably at least four, preferably at least five collagen peptides according to the invention.

According to this embodiment, a step of cleaving the collagen peptide expressed in step b) to obtain collagen peptides according to the invention, in particular collagen peptides with a molecular weight in a range from 0.18 to 10.0 kDa, in particular 0.18 to 5.0 kDa, in particular 1.1 to 5.0 kDa, takes place between steps b) and c). Accordingly, the expression system provided in step a) expresses a collagen peptide in step b), which is then cleaved so that several, preferably at least two, preferably at least three, preferably at least four, preferably at least five, collagen peptides according to the invention are obtained.

In a further preferred embodiment, the several, preferably at least two, preferably at least three, preferably at least four, preferably at least five, nucleotide sequences strung together are different nucleotide sequences. Particularly preferably, the plurality of, preferably at least two, preferably at least three, preferably at least four, preferably at least five, nucleotide sequences strung together are identical nucleotide sequences.

In a preferred embodiment of the present invention, the cleavage, in particular the enzymatic cleavage, of the collagen peptide expressed in step b), in particular the precursor collagen peptide, takes place using neutral, alkaline or acidic proteases.

The individual collagen peptides according to the invention of the expressed collagen peptide, in particular of the precursor collagen peptide, are preferably separated from one another by specific recognition sequences.

The specific recognition sequences are particularly preferably selected from the group consisting of factor Xa (Ile-(Glu/Asp)-Gly-Arg), TEV (Glu-Asn-Leu-Tyr-Phe-Gln-(Gly/Ser)), thrombin (Leu-Val-Pro-Arg-Gly-Ser), trypsin recognition sequence, papain recognition sequence.

In a preferred embodiment, according to the present invention, the collagen peptide is administered locally, in particular topically, or systemically, in particular enterally, preferably orally.

According to the invention, the term “biological activity” preferably denotes the ability of the collagen peptides according to the invention to stimulate the synthesis of extracellular matrix proteins, in particular the synthesis of collagen, proteoglycans and/or elastin, in cells, in particular osteoblasts, fibroblasts and/or chondrocytes. According to the invention, a “biological activity” is preferably present when through the incubation of cells, in particular osteoblasts, fibroblasts and/or chondrocytes, with the collagen peptides according to the invention, the synthesis of extracellular matrix proteins is stimulated, in particular the synthesis of collagen, proteoglycans and/or elastin, preferably measurable in at least one, preferably at least two, preferably in all of the in vitro tests shown in Examples 3 to 6, and the synthesis of extracellular matrix proteins is stimulated in osteoblasts, fibroblasts and chondrocytes, compared to untreated cells and/or cells treated with a biologically inactive agent, in particular untreated osteoblasts, fibroblasts and/or chondrocytes, or osteoblasts, fibroblasts and/or chondrocytes treated with a biologically inactive agent. In a preferred embodiment of the present invention, a “biologically inactive agent” is understood to mean 160 Bloom gelatin from pig skin or a 260 Bloom gelatin from bovine split.

According to the invention, “equal biological activity” is preferably understood to mean that the collagen peptides according to the invention stimulate the synthesis of extracellular matrix proteins, in particular the synthesis of collagen, proteoglycans and/or elastin, in cells, in particular osteoblasts, fibroblasts and/or chondrocytes, which is comparable to the stimulation of cells, in particular osteoblasts, fibroblasts and/or chondrocytes, which when incubated with a mixture of collagen peptides obtained from natural sources, can be measured in particular when incubated with a mixture of collagen peptides obtained from natural sources with a comparable average molecular weight, in particular with Verisol, produced according to EP 2640352 B1, Fortigel, produced according to WO 2010/149596 or Fortibone, produced according to WO 2014/072235 (EP 2916855 B1). According to the invention, a “comparable stimulation” preferably denotes a stimulation of the synthesis of extracellular matrix proteins, in particular the synthesis of collagen, proteoglycans and/or elastin, in cells, in particular osteoblasts, fibroblasts and/or chondrocytes, preferably measurable in at least one, preferably at least two, preferably in all of the in vitro tests shown in Examples 3 to 6 for stimulating the synthesis of extracellular matrix proteins in osteoblasts, fibroblasts and chondrocytes, which deviate by at most 2%, preferably at most 1.5%, preferably at most 1% from the stimulation of the synthesis of extracellular matrix proteins, in particular the synthesis of collagen, proteoglycans and/or elastin, in cells, in particular osteoblasts, fibroblasts and/or chondrocytes, which are produced by incubating the cells with a mixture of collagen peptides obtained from natural sources, in particular by incubating the cells with a mixture of collagen peptides obtained from natural sources with a comparable average molecular weight, in particular with Verisol, produced according to EP 2640352 B1, Fortigel, produced according to WO 2010/149596 or Fortibone, produced according to WO 2014/072235 (EP 2916855 B1).

According to the invention, the term “better biological activity” is preferably understood to mean that the collagen peptides according to the invention cause a stronger stimulation of the synthesis of extracellular matrix proteins, in particular the synthesis of collagen, proteoglycans and/or elastin, in cells, in particular osteoblasts, fibroblasts and/or chondrocytes, than a mixture of collagen peptides obtained from natural sources, in particular a mixture of collagen peptides obtained from natural sources with a comparable average molecular weight, in particular with Verisol, produced according to EP 2640352 B1, Fortigel, produced according to WO 2010/149596 or Fortibone, produced according to WO 2014/072235 (EP 2916855 B1). According to the invention, “better biological effectiveness” means an increase in the stimulation of the synthesis of extracellular matrix proteins, in particular the synthesis of collagen, proteoglycans and/or elastin, in cells, in particular osteoblasts, fibroblasts and/or chondrocytes, by the collagen peptides according to the invention by more than 2%, preferably at least 3%, preferably at least 4%, preferably at least 5%, compared to the stimulation of the synthesis of extracellular matrix proteins, in particular the synthesis of collagen, proteoglycans and/or elastin, in cells, in particular osteoblasts, fibroblasts and/or chondrocytes, by a mixture of collagen peptides obtained from natural sources, in particular with Verisol, produced according to EP 2640352 B1, Fortigel, produced according to WO 2010/149596 or Fortibone, produced according to WO 2014/072235 (EP 2916855 B1), wherein the increase in synthesis can be measured in at least one, preferably in at least two, preferably in all, of the in vitro tests shown in Examples 3 to 6 for stimulating the synthesis of extracellular matrix proteins in osteoblasts, fibroblasts and chondrocytes.

According to the invention, a “biological activity,” an “equal biological activity” or a “better biological activity” can accordingly be present if the collagen peptides according to the invention are capable of stimulating the synthesis of an extracellular matrix protein, preferably if at least the synthesis of one of the extracellular matrix proteins collagen, proteoglycan or elastin, preferably by two or three of these extracellular matrix proteins, is stimulated in cells, preferably in at least one of the cell types osteoblasts, fibroblasts or chondrocytes, preferably in two or three of these cell types. Accordingly, biological activity can also be present in one embodiment of the present invention if only the synthesis of an extracellular matrix protein is stimulated, in particular, for example, an extracellular matrix protein selected from the group consisting of collagen, proteoglycan and elastin, while for other extracellular matrix proteins there is no stimulation of the synthesis detected in the cells concerned. Biological activity, or the same or better biological activity, can also be present if stimulation of the synthesis of at least one extracellular matrix protein is found in only one cell type, in particular one cell type selected from the group consisting of osteoblasts, fibroblasts and chondrocytes, even if in other cell types such stimulation does not occur. It is preferably provided that the synthesis of more than one extracellular matrix protein, in particular of one or more of the explicitly mentioned extracellular matrix proteins selected from the group consisting of collagen, proteoglycan and elastin, is stimulated in cells, in particular in at least one of the cell types osteoblasts, fibroblasts or chondrocytes, particularly in two or three of these cell types.

According to the present invention, a “biological activity,” “equal biological activity” or “better biological activity” of the collagen peptides according to the invention in osteoblasts after incubation of the cells with the collagen peptides according to the invention can be determined in particular by determining the expression of the mRNA of the corresponding extracellular matrix proteins, in particular selected from the group consisting of collagen, proteoglycan and elastin, in comparison to the expression of the mRNA of the corresponding extracellular matrix proteins, in particular selected from the group consisting of collagen, proteoglycan and elastin, in suitable controls by means of real-time PCR, in particular by the in vitro test listed in Example 3.

According to the present invention, a “biological activity,” “equal biological activity” or “better biological activity” of the collagen peptides according to the invention in fibroblasts can, according to the present invention, be detected after incubation of the cells with the collagen peptides according to the invention by determining the expression of the mRNA of the corresponding extracellular matrix proteins, in particular selected from the group consisting of collagen, biglycan and versican, in comparison to the expression of the mRNA of the corresponding extracellular matrix proteins, in particular selected from the group consisting of collagen, biglycan and versican, in suitable controls using real-time PCR, in particular by the in vitro test listed in Example 4.

According to the present invention, a “biological effectiveness,” “equal biological effectiveness” or “better biological effectiveness” of the collagen peptides according to the invention in chondrocytes can be determined after incubation of the cells with the collagen peptides according to the invention by radioactive labeling and detection of the amount of synthesized radioactively labeled collagen and/or by Alcian blue staining and photometric determination of the glycosaminoglycans (GAG) of synthesized proteoglycans in each case in comparison with suitable controls, in particular by the in vitro tests listed in Example 5.

A “biological effectiveness,” an “equal biological effectiveness” or a “better biological effectiveness” of the collagen peptides according to the invention in osteoblasts, fibroblasts and chondrocytes, in particular in fibroblasts and chondrocytes, can be determined according to the present invention after incubation of the cells with the collagen peptides according to the invention through determination of the amount of synthesized extracellular matrix proteins, in particular selected from the group consisting of collagen, proteoglycan and elastin, particularly preferably selected from the group consisting of collagen and proteoglycan, in comparison to the determination of the amount of synthesized extracellular matrix proteins, in particular selected from the group consisting of collagen, proteoglycan and elastin, particularly preferably selected from the group consisting of collagen and proteoglycan, in suitable controls by means of photometric determination of the amount of synthesized extracellular matrix proteins labeled with dye, in particular selected from the group consisting of collagen, proteoglycan and elastin, particularly preferably selected from the group consisting of collagen and proteoglycan, in particular by the in vitro tests listed in Example 6.

According to the invention, the term “biological effectiveness in at least one, preferably in at least two, preferably in all, of the in vitro tests shown in Examples 3 to 6 for stimulating the synthesis of extracellular matrix proteins in osteoblasts, fibroblasts and chondrocytes” means that the biological activity of the collagen peptides according to the invention can be shown with one of the assays explicitly carried out in Examples 3 to 6. The individual process parameters listed in Examples 3 to 6 can optionally be varied in accordance with expert understanding without influencing the fundamental significance of the test results. In a preferred embodiment of the present invention, the collagen peptides according to the invention show a biological activity in at least one, preferably in at least two, preferably in all of the in vitro tests shown in Examples 3 to 6 for stimulating the synthesis of extracellular matrix proteins in osteoblasts, fibroblasts, and chondrocytes under exactly the method parameters listed in these examples. Furthermore, the biological effectiveness of the collagen peptides according to the invention can also be demonstrated with further tests known to the person skilled in the art, in particular in vitro tests, preferably in vitro tests for stimulating the synthesis of extracellular matrix proteins in osteoblasts, fibroblasts and chondrocytes.

The term “stimulation of the synthesis of extracellular matrix proteins” according to the invention means to stimulate the biosynthesis of at least one protein of the extracellular matrix in cells, preferably in osteoblasts, fibroblasts and/or chondrocytes, and/or stimulate the biosynthesis of at least one mRNA encoding protein of the extracellular matrix in cells, preferably in osteoblasts, fibroblasts and/or chondrocytes, understood through exogenous influences, in particular through the collagen peptides according to the invention. In particular, the term “stimulation of the synthesis of extracellular matrix proteins” according to the invention means an increase in the amount of at least one protein of the extracellular matrix secreted by cells, preferably osteoblasts, fibroblasts and/or chondrocytes, and/or an increase in the synthesized amount in cells, preferably in osteoblasts, fibroblasts and/or chondrocytes, of at least one mRNA encoding a protein of the extracellular matrix by exogenous influences, in particular by the collagen peptides according to the invention.

The term “collagen” in connection with the present invention is understood in a manner customary in the art, in particular as defined, for example, in WO 01/34646. In a preferred embodiment, the term “collagen” relates to collagen types I to XXVII. In a further preferred embodiment, the term “collagen” is understood to mean a peptide that includes the sequence glycine-proline, glycine-4-hydroxyproline or glycine-X-4-hydroxyproline, preferably the repetitive motif (Gly-X-Y)ₙ, where X and Y may be any amino acid, preferably proline and 4-hydroxyproline. The term “collagen” is particularly preferably understood to mean a peptide having the repetitive motif (Gly-Pro-Y)ₙ and/or (Gly-X-Hyp)ₘ, where X and Y may be any amino acid.

In connection with the present invention, the term “gelatin” is preferably understood in a manner customary in the art, in particular as defined, for example, in WO 01/34646.

In connection with the present invention, the term “collagen peptide” is understood to mean a peptide which has an amino acid sequence occurring in collagen as defined above, the peptide being at least one dipeptide, preferably an oligopeptide or a polypeptide. The collagen peptide can in particular be present in chemically modified form, in particular hydroxylated and/or glycosylated form, or it can be unmodified.

According to the invention, the term “function-maintaining sequence modification” is understood to mean the modification of a given amino acid sequence, in particular the replacement, addition and/or deletion of one or more amino acids, which leads to an amino acid sequence that deviates from the given amino acid sequence, but retains the modified amino acid sequence for the given amino acid sequence characteristic function, in particular its biological activity.

A “function-preserving sequence modification” is preferably understood to mean a modification of a given amino acid sequence in which the function characteristic of the given amino acid sequence, in particular a biological activity, in particular a biological activity with regard to connective tissue, in particular a biological activity according to the in vitro tests shown in Examples 3 to 6, is maintained for stimulating the synthesis of extracellular matrix proteins in osteoblasts, fibroblasts and chondrocytes to at least 50%, preferably at least 60%, preferably at least 70%, preferably at least 80%, preferably at least 90%, preferably at least 95%, preferably 100%. According to the invention, a “function-preserving sequence modification” is more preferably understood to mean a modification of a given amino acid sequence in which the modified amino acid sequence has at least 50%, preferably at least 55%, preferably at least 60%, preferably at least 65%, preferably at least 70%, preferably at least 75%, preferably at least 80%, preferably at least 85%, preferably at least 90%, sequence homology to the given amino acid sequence.

Particularly preferred in a “function-maintaining sequence modification” in connection with the present invention is a modification of a given amino acid sequence in which one or more amino acids with certain chemico-physical properties have been replaced by one or more amino acids with the same or similar chemo-physical properties, in particular for example an amino acid with a non-polar side chain (for example Ala, Val, Met, Leu, Ile, Pro, Trp, Phe) is replaced by another amino acid with a non-polar side chain (for example Ala, Val, Met, Leu, Ile, Pro, Trp, Phe), an amino acid with a polar, neural side chain (for example Tyr, Thr, Gln, Gly, Ser, Cys, Asn) is replaced by another amino acid with a polar, neural side chain (for example Tyr, Thr, Gln, Gly, Ser, Cys, Asn), an amino acid with an acidic side chain (e.g. Glu, Asp) is replaced by another amino acid with an acidic side chain (for example Glu, Asp) and/or an amino acid with a basic side chain (for example Lys, Arg, His) is replaced by another amino acid with a basic side chain (for example Lys, Arg, His). According to this embodiment, the chemico-physical properties of a given amino acid sequence are retained in the “function-maintaining sequence modification” or only change slightly.

In a further embodiment it can be provided that the “function-maintaining sequence modification” consists in that at least one amino acid of a given amino acid sequence, in particular a naturally occurring amino acid sequence, preferably at least one non-essential amino acid, in particular Ala, Asn, Asp, Glu, Ser, of the given amino acid sequence, in particular the naturally occurring amino acid sequence, is replaced by at least one very specific amino acid, in particular at least one essential amino acid, in particular Ile, Leu, Lys, Met, Phe, Thr, Trp, Val, His, Cys, Tyr, particularly preferably Trp, wherein the function characteristic of the given amino acid sequence, in particular the naturally occurring amino acid sequence, in particular the biological activity, in particular the biological activity with regard to connective tissue, in particular the biological activity according to the in vitro stimulation tests shown in Examples 3 to 6 for stimulating the synthesis of extracellular matrix proteins in osteoblasts, fibroblasts and chondrocytes is maintained to an extent of at least 50%, preferably at least 60%, preferably at least 70%, preferably at least 80%, preferably at least 90%, preferably at least 95%, preferably 100%. The synthetic or recombinant collagen peptides, in particular the synthetically or recombinantly produced collagen peptides according to SEQ ID no. 23, 27, 28, are each a “function-maintaining sequence modification” of the synthetic or recombinant collagen peptide, in particular of the synthetically or recombinantly produced collagen peptide, according to SEQ ID No. 7, wherein non-essential amino acids have been replaced by essential amino acids. The synthetic or recombinant collagen peptides, in particular the synthetically or recombinantly produced collagen peptides, according to SEQ ID No. 24, 29 and 30, are each a “function-preserving sequence modification” of the synthetic or recombinant collagen peptide, in particular of the synthetically or recombinantly produced collagen peptide, according to SEQ ID No. 22, with non-essential amino acids being replaced by essential amino acids.

According to the invention, a “function-maintaining sequence modification” is also understood to mean the modification of a given amino acid sequence, in particular a naturally occurring amino acid sequence, which consists in the given amino acid sequence, in particular the naturally occurring amino acid sequence, having at least one amino acid, preferably at least one essential amino acid, in particular Ile, Leu, Lys, Met, Phe, Thr, Trp, Val, His, Cys, Tyr, particularly preferably Trp, added to it, wherein the function characteristic of the given amino acid sequence, in particular the naturally occurring amino acid sequence, in particular the biological activity, in particular the biological activity with regard to connective tissue, in particular the biological activity according to the in vitro tests shown in Examples 3 to 6 for stimulating the synthesis of extracellular matrix proteins in osteoblasts, fibroblasts and chondrocytes is maintained to an extent of at least 50%, preferably at least 60%, preferably at least 70%, preferably at least 80%, preferably at least 90%, preferably at least 95%, preferably 100%. In this case, it may be provided according to the invention that the at least one amino acid, preferably the at least one essential amino acid, in particular Ile, Leu, Lys, Met, Phe, Thr, Trp, Val, His, Cys, Tyr, particularly preferably Trp, has been added N-terminally, C-terminally and/or within the amino acid sequence.

In connection with the present invention, the term “amino acid modification” denotes a chemical change in one or more amino acids that took place before, after or during the synthesis of the collagen peptide while maintaining the original amino acid skeleton, in particular one or more proteinogenic amino acids of the collagen peptide. The term thus encompasses both the use of chemically modified amino acids for the synthesis of the collagen peptide according to the invention and the chemical modification of the amino acids after or during the synthesis of the collagen peptide Amino acid modifications typical for collagen peptides are, in particular, hydroxylations on proline and lysine residues and glycosylations on hydroxylated lysine residues. According to the invention, however, the term also includes other chemical changes in amino acids, such as phosphorylations, N-glycosylations, acetylations, methylations, myristoylations.

In connection with the present invention, a “synthetic or recombinant collagen peptide” or a “synthetically or recombinantly produced collagen peptide” is understood to mean a collagen peptide obtained by chemical synthesis, in particular solid phase synthesis, or by biotechnological recombinant production using an expression system. According to the invention, the “synthetic collagen peptide” or the “synthetically produced collagen peptide” and the “recombinant collagen peptide” or the “recombinantly produced collagen peptide” have in common that they are not obtained from natural sources.

In connection with the present invention, the term “recombinant DNA” denotes an artificially produced or manipulated DNA molecule, which has been produced in vitro by means of genetic engineering methods. In a preferred embodiment, the recombinant DNA is composed of components from different original organisms.

In connection with the present invention, the term “expression cassette” is understood to mean a DNA segment which is responsible for the transcription of the information encoded in this segment into an RNA, in particular into an mRNA, and includes at least one promoter and one protein-encoding nucleotide sequence, usually has at least one promoter, at least one protein-encoding nucleotide sequence, and optionally a terminator.

In connection with the present invention, a “nucleotide sequence” is understood to mean the sequence of the nucleotides of a nucleic acid, in particular a nucleic acid strand, in particular a DNA or RNA strand. A “nucleotide sequence” is therefore to be understood both as an informational unit and as the DNA or RNA strand physically manifesting this information.

In connection with the present invention, an “expression system” is understood to mean a system in which a targeted and controlled protein biosynthesis may take place. According to the invention, the term “expression system” encompasses both cell-free expression systems in which the components necessary for protein biosynthesis are not present within a cell, i.e., protein synthesis takes place outside of a cell, as well as cell-based expression systems in which protein biosynthesis takes place within a living cell. In connection with the present invention, a cell-free expression system is preferably a lysate or an extract from E. coli, insect cells, wheat germ, tobacco cells or mammalian cells, in particular CHO cells or reticulocytes from rabbits, which contains the components necessary for protein biosynthesis, in particular a translation and a transcription system. If a cell-free expression system is used in one of the methods according to the present invention, the term “culturing” is synonymous with “incubating.”

In connection with the present invention, a “host cell” is understood to mean a living cell which is capable of expressing peptides or proteins encoded in foreign DNA, in particular in recombinant DNA.

According to the invention, the terms “obtaining the collagen peptide” according to method step c) is understood to mean a method known to the person skilled in the art for isolating the collagen peptide from a composition containing multiple components by means of known isolation methods such as, for example, centrifugation methods, in particular differential centrifugation and/or density gradient centrifugation, chromatographic methods, in particular gel filtration, ion exchange, affinity and/or high-performance liquid chromatography, electrophoresis methods, filtration methods and/or extraction methods, wherein an enrichment and purification of the relevant component from the composition containing multiple components may be achieved, preferably via the sequential application of multiple isolation methods.

According to the invention, “conditions which enable the expression of the collagen peptide” are understood to mean conditions, such as, in particular, temperature, pressure, time, light and the presence or absence of inducers and/or repressors, which activate or intensify an expression of the collagen peptide. In one preferred embodiment, the expression of the collagen peptide takes place in the context of a high cell density fermentation, in particular under high pressure, preferably high pressure air. The specific conditions which enable an expression of the collagen peptide are known to the person skilled in the art and depend on the expression system used and the expression cassette used, in particular, on the promoter contained therein. The expression of the collagen peptide may be a constitutive or inducible expression, depending on the structure of the expression cassette.

In connection with the present invention, the terms “comprising” and “including” are understood to mean that in addition to the elements explicitly covered by these terms, further elements that are not explicitly mentioned may be added. In connection with the present invention, these terms are also understood to mean that only the explicitly mentioned elements are included and no further elements are present. In this particular embodiment, the meaning of the terms “comprising” and “including” is synonymous with the term “consisting of.” In addition, the terms “comprising” and “including” also include compositions which, in addition to the explicitly named elements, also contain other elements not mentioned, but which are functionally and qualitatively subordinate. In this embodiment, the terms “comprising” and “including” are synonymous with the term “consisting essentially of.”

If, in connection with the present invention, the first and second decimal places or the second decimal place are/is not specified, these are/is to be set to 0.

In connection with the present invention, the term “and/or” is understood to mean that all members of a group which are connected by the term “and/or” are disclosed both as alternatives to one another and also cumulatively to one another in any combination. For the expression “A, B and/or C,” this means that the following disclosure content is to be understood to mean: a) A or B or C orb) (A and B) or c) (A and C) or d) (B and C) or e) (A and B and C).

Further preferred embodiments result from the dependent claims.

The invention is described below without restricting the general inventive concept on the basis of exemplary sequences, figures, and embodiments.

In the following:

SEQ ID No. 1 denotes the amino acid sequence Leu-Thr-Gly-Pro-Ile-Gly-Pro-Pro-Gly-Pro-Ala of a non-hydroxylated collagen peptide comprising 11 amino acids.

SEQ ID No. 2 denotes the amino acid sequence Gly-Ala-Pro-Gly-Lys-Asp-Gly-Val-Arg-Gly-Leu-Thr-Gly-Pro-Ile-Gly-Pro-Pro-Gly-Pro-Ala-Gly-Ala-Pro-Gly-Asp-Lys-Gly-Glu-Ala-Gly-Pro-Ser of a non-hydroxylated collagen peptide comprising 33 amino acids.

SEQ ID No. 3 denotes the amino acid sequence Gly-Ala-Pro-Gly-Lys-Asp-Gly-Val-Arg-Gly-Leu-Thr-Gly-Pro-Ile-Gly-Pro-Pro-Gly-Pro-Ala-Gly-Ala-Pro of a non-hydroxylated collagen peptide comprising 24 aminos acids.

SEQ ID No. 4 denotes the amino acid sequence Gly-Ala-Pro-Gly-Lys-Asp-Gly-Val-Arg-Gly-Leu-Thr-Gly-Pro-Ile-Gly-Pro-Pro-Gly-Pro-Ala-Gly-Ala-Arg of a non-hydroxylated collagen peptide comprising 24 amino acids.

SEQ ID No. 5 denotes the amino acid sequence Gly-Ala-Pro-Gly-Pro-Pro-Gly-Pro-Pro-Gly-Ala-Arg-Gly-Gln-Ala-Gly-Val-Met-Gly-Phe-Pro-Gly-Pro-Lys of a non-hydroxylated collagen peptide comprising 24 amino acids.

SEQ ID No. 6 denotes the amino acid sequence Leu-Thr-Gly-Pro-Ile-Gly-Pro-4Hyp-Gly-Pro-Ala of a hydroxylated collagen peptide comprising 11 amino acids.

SEQ ID No. 7 denotes the amino acid sequence Gly-Ala-4Hyp-Gly-Lys-Asp-Gly-Val-Arg-Gly-Leu-Thr-Gly-Pro-Ile-Gly-Pro-4Hyp-Gly-Pro-Ala-Gly-Ala-4Hyp-Gly-Asp-Lys-Gly-Glu-Ala-Gly-Pro-Ser of a hydroxylated collagen peptide comprising 33 amino acids.

SEQ ID No. 8 denotes the amino acid sequence Gly-Ala-4Hyp-Gly-Lys-Asp-Gly-Val-Arg-Gly-Leu-Thr-Gly-Pro-Ile-Gly-Pro-4Hyp-Gly-Pro-Ala-Gly-Ala-4Hyp of a hydroxylated collagen peptide comprising 24 aminos acids.

SEQ ID No. 9 denotes the amino acid sequence Gly-Ala-4Hyp-Gly-Lys-Asp-Gly-Val-Arg-Gly-Leu-Thr-Gly-Pro-Ile-Gly-Pro-4Hyp-Gly-Pro-Ala-Gly-Ala-Arg of a hydroxylated collagen peptide comprising 24 amino acids.

SEQ ID No. 10 denotes the amino acid sequence Gly-Ala-4Hyp-Gly-Pro-Pro-Gly-Pro-4Hyp-Gly-Ala-Arg-Gly-Gln-Ala-Gly-Val-Met-Gly-Phe-4Hyp-Gly-Pro-Lys of a hydroxylated collagen peptide comprising 24 amino acids.

SEQ ID No. 11 denotes the amino acid sequence Gly-Ala-Pro-Gly-Lys-Asp-Gly-Val-Arg-Gly-Leu of a non-hydroxylated collagen peptide comprising 11 amino acids.

SEQ ID No. 12 denotes the amino acid sequence Gly-Ala-Pro-Gly-Lys-Asp-Gly-Val-Arg-Gly-Leu-Thr-Gly-Pro-Ile-Gly-Pro-Pro-Gly-Pro-Ala-Gly-Ala of a non-hydroxylated collagen peptide comprising 23 amino acids.

SEQ ID No. 13 denotes the amino acid sequence Gly-Ala-Pro-Gly-Lys-Asp-Gly-Val-Arg-Gly-Leu-Thr-Gly-Pro-Ile-Gly-Pro-Pro-Gly-Pro-Ala-Gly-Ala-Pro-Gly-Asp-Lys-Gly-Glu-Ala-Gly-Pro-Ser-Gly-Pro-Ala-Gly-Pro-Thr-Gly-Ala-Arg-Gly-Ala-Pro-Gly-Asp-Arg-Gly-Glu-Pro-Gly-Pro-Pro-Gly-Pro-Ala-Gly of a non-hydroxylated collagen peptide comprising 58 amino acids.

SEQ ID No. 14 denotes the amino acid sequence Gly-Ala-Pro-Gly-Lys-Asp-Gly-Val-Arg-Gly-Leu-Thr-Gly-Pro-Ile-Gly-Pro-Pro-Gly-Pro-Ala-Gly-Ala-Pro-Gly-Asp-Lys-Gly-Glu-Ala-Gly-Pro-Ser-Gly-Pro-Ala-Gly-Pro-Thr-Gly-Ala-Arg-Gly-Ala-Pro-Gly-Asp-Arg-Gly-Glu-Pro-Gly-Pro-Pro-Gly-Pro-Ala-Gly-Phe-Ala-Gly-Pro-Pro-Gly-Ala-Asp-Gly-Gln-Pro-Gly-Ala-Lys-Gly-Glu-Pro-Gly-Asp-Ala-Gly-Ala-Lys-Gly-Asp-Ala-Gly-Pro-Pro-Gly-Pro-Ala of a non-hydroxylated collagen peptide comprising 90 amino acids.

SEQ ID No. 15 denotes the amino acid sequence Lys-Gly-Ala-Pro-Gly-Ala-Asp-Gly-Pro-Ala-Gly-Ala-Pro-Gly-Thr-Pro-Gly-Pro-Gln of a non-hydroxylated collagen peptide comprising 19 amino acids.

SEQ ID No. 16 denotes the amino acid sequence Gly-Pro-Pro-Gly-Pro-Ala-Gly-Glu-Lys-Gly-Ala-Pro-Gly-Ala-Asp-Gly-Pro-Ala-Gly-Ala-Pro-Gly-Thr-Pro-Gly-Pro-Gln-Gly-Ile-Ala-Gly-Gln-Arg-Gly-Val of a non-hydroxylated collagen peptide comprising 35 amino acids.

SEQ ID No. 17 denotes the amino acid sequence Gly-Ala-4Hyp-Gly-Lys-Asp-Gly-Val-Arg-Gly-Leu of a hydroxylated collagen peptide comprising 11 amino acids.

SEQ ID No. 18 denotes the amino acid sequence Gly-Ala-4Hyp-Gly-Lys-Asp-Gly-Val-Arg-Gly-Leu-Thr-Gly-Pro-Ile-Gly-Pro-4Hyp-Gly-Pro-Ala-Gly-Ala of a hydroxylated collagen peptide comprising 23 amino acids.

SEQ ID No. 19 denotes the amino acid sequence Gly-Ala-4Hyp-Gly-Lys-Asp-Gly-Val-Arg-Gly-Leu-Thr-Gly-Pro-Ile-Gly-Pro-4Hyp-Gly-Pro-Ala-Gly-Ala-4Hyp-Gly-Asp-Lys-Gly-Glu-Ala-Gly-Pro-Ser-Gly-Pro-Ala-Gly-Pro-Thr-Gly-Ala-Arg-Gly-Ala-4Hyp-Gly-Asp-Arg-Gly-Glu-4Hyp-Gly-Pro-4Hyp-Gly-Pro-Ala-Gly of a hydroxylated collagen peptide comprising 58 amino acids.

SEQ ID No. 20 denotes the amino acid sequence Gly-Ala-4Hyp-Gly-Lys-Asp-Gly-Val-Arg-Gly-Leu-Thr-Gly-Pro-Ile-Gly-Pro-4Hyp-Gly-Pro-Ala-Gly-Ala-4Hyp-Gly-Asp-Lys-Gly-Glu-Ala-Gly-Pro-Ser-Gly-Pro-Ala-Gly-Pro-Thr-Gly-Ala-Arg-Gly-Ala-4Hyp-Gly-Asp-Arg-Gly-Glu-4Hyp-Gly-Pro-4Hyp-Gly-Pro-Ala-Gly-Phe-Ala-Gly-Pro-4Hyp-Gly-Ala-Asp-Gly-Gln-4Hyp-Gly-Ala-Lys-Gly-Glu-Pro-Gly-Asp-Ala-Gly-Ala-Lys-Gly-Asp-Ala-Gly-Pro-Pro-Gly-Pro-Ala of a hydroxylated collagen peptide comprising 90 amino acids.

SEQ ID No. 21 denotes the amino acid sequence Lys-Gly-Ala-4Hyp-Gly-Ala-Asp-Gly-Pro-Ala-Gly-Ala-4Hyp-Gly-Thr-Pro-Gly-Pro-Gln of a hydroxylated collagen peptide comprising 19 amino acids.

SEQ ID No. 22 denotes the amino acid sequence Gly-Pro-4Hyp-Gly-Pro-Ala-Gly-Glu-Lys-Gly-Ala-4Hyp-Gly-Ala-Asp-Gly-Pro-Ala-Gly-Ala-4Hyp-Gly-Thr-Pro-Gly-Pro-Gln-Gly-Ile-Ala-Gly-Gln-Arg-Gly-Val of a hydroxylated collagen peptide comprising 35 amino acids.

SEQ ID No. 23 denotes the amino acid sequence Gly-Val-4Hyp-Gly-Lys-Tyr-Gly-Val-Arg-Gly-Leu-Thr-Gly-Pro-Ile-Gly-Pro-4Hyp-Gly-Pro-Leu-Gly-Ile-4Hyp-Gly-Trp-Lys-Gly-Phe-Val-Gly-Pro-Thr of a hydroxylated collagen peptide comprising 33 amino acids.

SEQ ID No. 24 denotes the amino acid sequence Gly-Pro-4Hyp-Gly-Pro-Val-Gly-Thr-Lys-Gly-Ile-4Hyp-Gly-Val-Tyr-Gly-Pro-Leu-Gly-Ile-4Hyp-Gly-Thr-Pro-Gly-Pro-Trp-Gly-Ile-Leu-Gly-Thr-Lys-Arg-Gly-Val of a hydroxylated collagen peptide comprising 36 amino acids.

SEQ ID No. 25 denotes the amino acid sequence Gly-Ala-4Hyp-Gly-Lys-Asp-Gly-Val-Arg-Gly-Leu-Thr-Gly-4Hyp-Ile-Gly-4Hyp-4Hyp-Gly-4Hyp-Ala-Gly-Ala-4Hyp-Gly-Asp-Lys-Gly-Glu-Ala-Gly-4Hyp-Ser of a hydroxylated collagen peptide comprising 33 amino acids.

SEQ ID No. 26 denotes the amino acid sequence His-His-His-His-His-His-Gly-Ala-4Hyp-Gly-Lys-Asp-Gly-Val-Arg-Gly-Leu-Thr-Gly-Pro-Ile-Gly-Pro-4Hyp-Gly-Pro-Ala-Gly-Ala-4Hyp-Gly-Asp-Lys-Gly-Glu-Ala-Gly-Pro-Ser of a hydroxylated collagen peptide comprising 39 amino acids.

SEQ ID No. 27 denotes the amino acid sequence Gly-Leu-4Hyp-Gly-Lys-Met-Gly-Val-Phe-Gly-Leu-Thr-Gly-Pro-Ile-Gly-Pro-4Hyp-Gly-Pro-Trp-Gly-Val-4Hyp-Gly-His-Lys-Gly-Tyr-Leu-Gly-Pro-Thr of a hydroxylated collagen peptide comprising 33 amino acids.

SEQ ID No. 28 denotes the amino acid sequence Gly-Leu-4Hyp-Gly-Lys-Tyr-Gly-Val-His-Gly-Leu-Thr-Gly-Pro-Leu-Gly-Pro-4Hyp-Gly-Pro-Met-Gly-Ile-4Hyp-Gly-Trp-Lys-Gly-Phe-Val-Gly-Pro-Thr of a hydroxylated collagen peptide comprising 33 amino acids.

SEQ ID No. 29 denotes the amino acid sequence Gly-Pro-4Hyp-Gly-Pro-Leu-Gly-Met-Lys-Gly-Leu-4Hyp-Gly-Val-Trp-Gly-Pro-Phe-Gly-Leu-4Hyp-Gly-Thr-Pro-Gly-Pro-His-Gly-Ile-Thr-Gly-Tyr-Lys-Gly-Val of a hydroxylated collagen peptide comprising 35 amino acids.

SEQ ID No. 30 denotes the amino acid sequence Gly-Pro-4Hyp-Gly-Pro-Val-Gly-Thr-Lys-Gly-Leu-4Hyp-Gly-Phe-Tyr-Gly-Pro-Leu-Gly-Ile-4Hyp-Gly-His-Pro-Gly-Pro-Trp-Gly-Met-Leu-Gly-Thr-Lys-Gly-Val of a hydroxylated collagen peptide comprising 35 amino acids.

In the following:

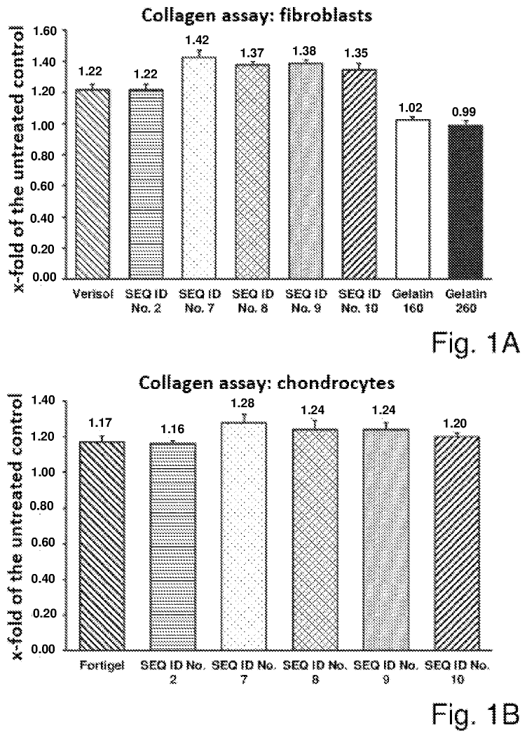

FIG. 1A shows the stimulation of the collagen synthesis of human dermal fibroblasts by the collagen peptides according to the invention of SEQ ID No. 2, SEQ ID No. 7, SEQ ID No. 8, SEQ ID No. 9, SEQ ID No. 10 in comparison to a mixture of collagen peptides obtained from natural sources with comparable average molecular weight (Verisol) and in comparison to two different gelatin controls (160 Bloom gelatin from pork rind (Gelatin 160), 260 Bloom gelatin from bovine split (Gelatin 260)) according to Example 6. The figure shows the factor for the quantitative determination of collagen synthesis compared to an untreated sample. The error bars show the standard error (SEM).

FIG. 1B shows the stimulation of the collagen synthesis of human chondrocytes by the collagen peptides according to the invention of SEQ ID No. 2, SEQ ID No. 7, SEQ ID No. 8, SEQ ID No. 9, SEQ ID No. 10 in comparison to a mixture of collagen peptides obtained from natural sources with a comparable average molecular weight (Fortigel) according to Example 6. The figure shows the factor for the quantitative determination of collagen synthesis compared to an untreated sample. The error bars show the standard error (SEM).

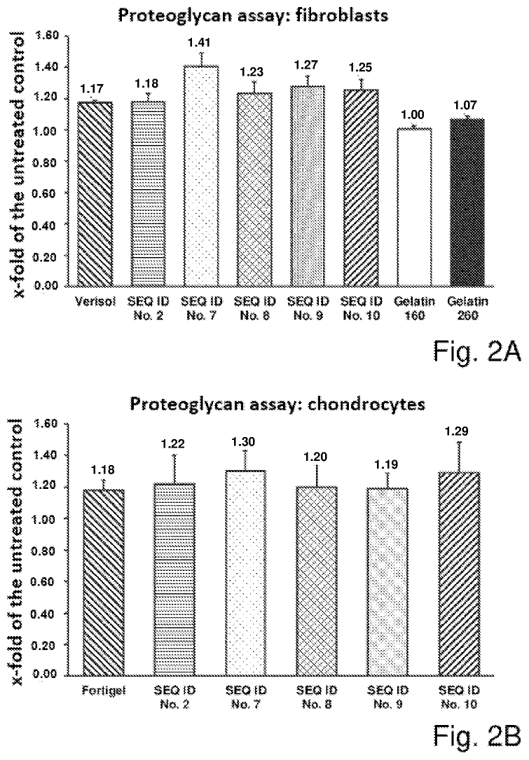

FIG. 2A shows the stimulation of the synthesis of proteoglycans from human dermal fibroblasts by the collagen peptides according to the invention of SEQ ID No. 2, SEQ ID No. 7, SEQ ID No. 8, SEQ ID No. 9, SEQ ID No. 10 in comparison to a mixture of collagen peptides obtained from natural sources with comparable average molecular weight (Verisol) and in comparison to two different gelatin controls (160 Bloom gelatin from pork rind (Gelatin 160), 260 Bloom gelatin from bovine split (Gelatin 260)) according to Example 6. The figure shows the factor for the quantitative determination of the synthesis of proteoglycans compared to an untreated sample. The error bars show the standard error (SEM).

FIG. 2B shows the stimulation of the synthesis of proteoglycans from human chondrocytes by the collagen peptides according to the invention of SEQ ID No. 2, SEQ ID No. 7, SEQ ID No. 8, SEQ ID No. 9, SEQ ID No. 10 in comparison to a mixture of collagen peptides obtained from natural sources with a comparable average molecular weight (Fortigel) according to Example 6. The figure shows the factor for the quantitative determination of collagen synthesis compared to an untreated sample. The error bars show the standard error (SEM).

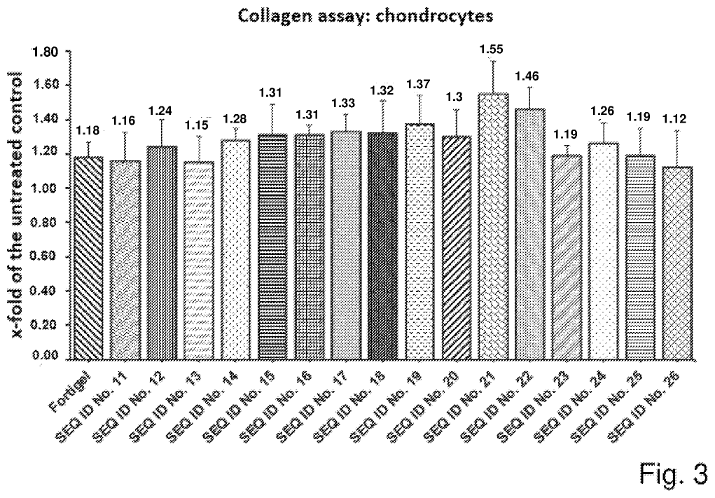

FIG. 3 shows the stimulation of the collagen synthesis of human chondrocytes by the collagen peptides according to the invention of SEQ ID No. 11 to SEQ ID No. 26 in comparison to a mixture of collagen peptides obtained from natural sources with a comparable average molecular weight (Fortigel). The figure shows the factor for the quantitative determination of collagen synthesis compared to an untreated sample. The error bars show the standard error (SEM).

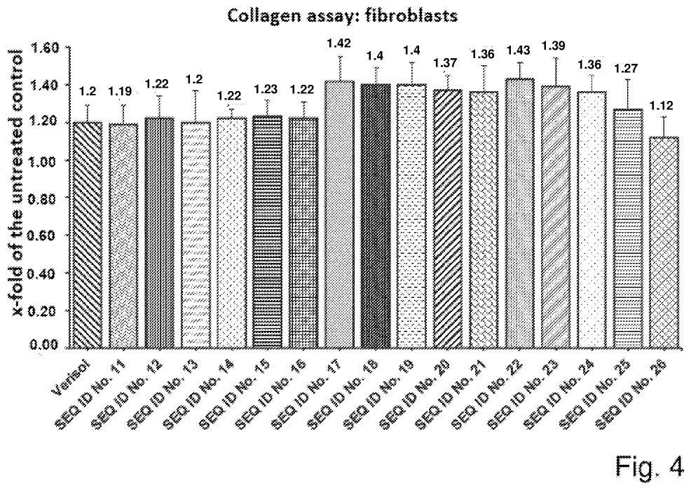

FIG. 4 shows the stimulation of the collagen synthesis of human dermal fibroblasts by the collagen peptides according to the invention of SEQ ID No. 11 to SEQ ID No. 26 in comparison to a mixture of collagen peptides obtained from natural sources with a comparable average molecular weight (Verisol). The figure shows the factor for the quantitative determination of collagen synthesis compared to an untreated sample. The error bars show the standard error (SEM).

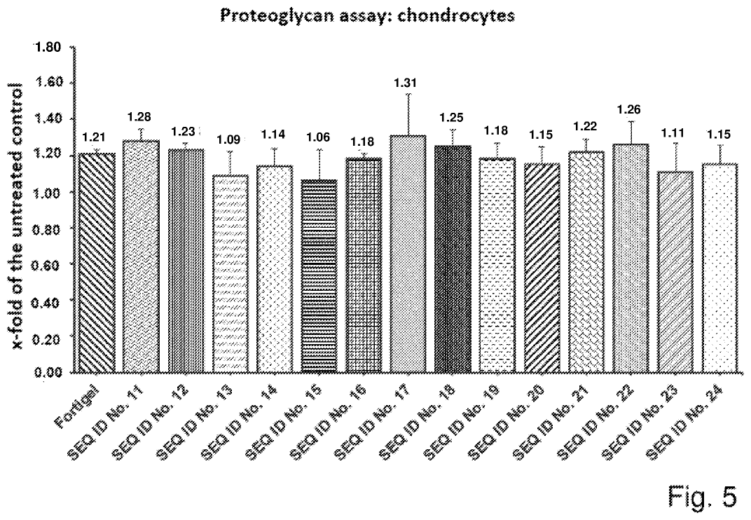

FIG. 5 shows the stimulation of the synthesis of proteoglycans from human chondrocytes by the collagen peptides according to the invention of SEQ ID No. 11 to SEQ ID No. 24 in comparison to a mixture of collagen peptides obtained from natural sources with a comparable average molecular weight (Fortigel). The figure shows the factor for the quantitative determination of collagen synthesis compared to an untreated sample. The error bars show the standard error (SEM).

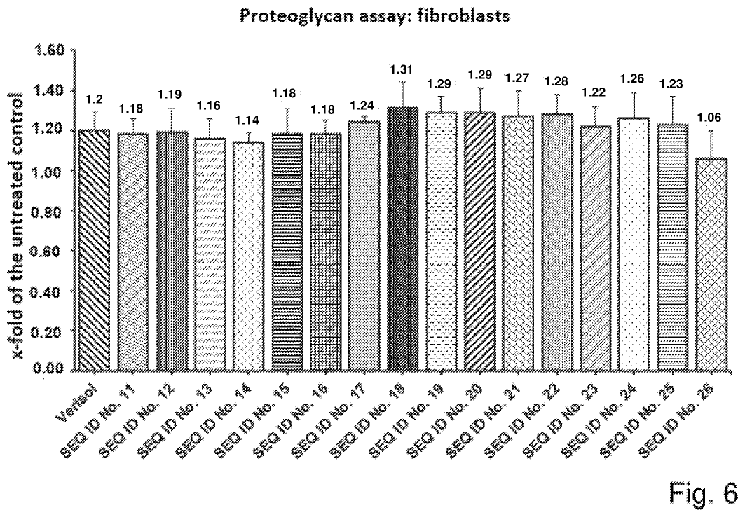

FIG. 6 shows the stimulation of the synthesis of proteoglycans of human dermal fibroblasts by the collagen peptides according to the invention of SEQ ID No. 11 to SEQ ID No. 26 in comparison to a mixture of collagen peptides obtained from natural sources with a comparable average molecular weight (Verisol). The figure shows the factor for the quantitative determination of collagen synthesis compared to an untreated sample. The error bars show the standard error (SEM).

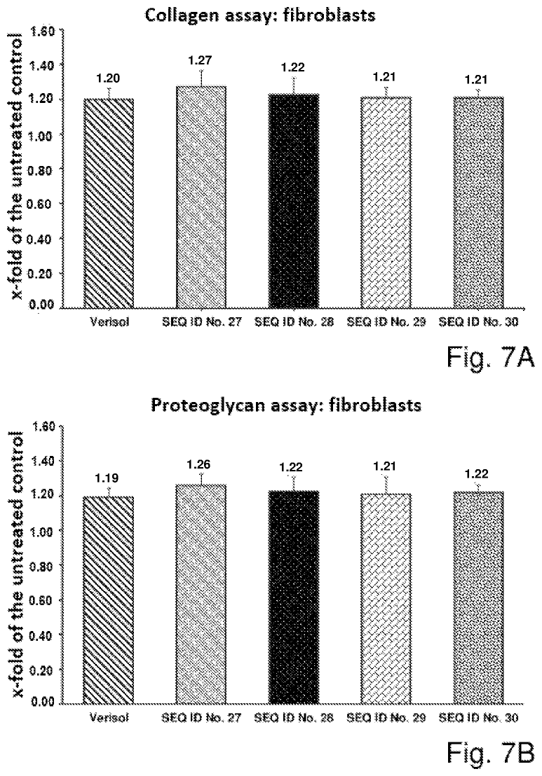

FIG. 7A shows the stimulation of the collagen synthesis of human dermal fibroblasts by the collagen peptides according to the invention of SEQ ID NO. 27 to SEQ ID No. 30 in comparison to a mixture of collagen peptides obtained from natural sources with a comparable average molecular weight (Verisol). The figure shows the factor for the quantitative determination of collagen synthesis compared to an untreated sample. The error bars show the standard error (SEM).

FIG. 7B shows the stimulation of the synthesis of proteoglycans of human dermal fibroblasts by the collagen peptides according to the invention of SEQ ID No. 27 to SEQ ID No. 30 in comparison to a mixture of collagen peptides obtained from natural sources with a comparable average molecular weight (Verisol). The figure shows the factor for the quantitative determination of collagen synthesis compared to an untreated sample. The error bars show the standard error (SEM).

### EXAMPLES

### Example 1—Solid Phase Synthesis

Collagen peptides according to the invention of the amino acid sequences SEQ ID No. 1 to SEQ ID No. 10 and SEQ ID No. 27 to SEQ ID No. 30 were obtained by solid phase synthesis (Merrifield synthesis) on a polystyrene resin.

### Example 2—Recombinant Production

Further collagen peptides, in particular the collagen peptides of the amino acid sequences SEQ ID No. 1 to SEQ ID No. 10, were also obtained by recombinant expression in Pichia pastoris.

### Example 3—Bone Health

To analyze the biological activity of the collagen peptide according to the invention in terms of maintaining bone health and the prophylaxis and treatment of bone diseases, its stimulating effect on the synthesis of extracellular matrix proteins and enzymes that play a role in the structure and mineralization of the matrix is examined via osteoblasts in vitro. This is done by determining the expression of the corresponding mRNA by means of real-time PCR and a semi-quantitative evaluation (based on a control without collagen peptide).

For this purpose, human osteoblasts are first isolated from knee joints by incubating bone material under vigorous agitation at 37° C. for 1 h in Hanks' solution, supplemented with 7 mg/ml hyaluronidase type I and III-S and 5 mg/ml pronase. The digestion is then continued at 37° C. for 3-5 h in Hanks' solution supplemented with 16 mg/ml collagenase type CLS IV. The primary osteoblasts obtained are cultivated after enzymatic digestion in Ham's F12 medium, supplemented with 10% fetal calf serum, 20 U/ml penicillin-streptomycin, 50 μg/ml partricin, 0.05 mg/ml ascorbic acid and 0.15 mg/ml glutamine. Alternatively, primary osteoblasts (Article No. C-12760; 2019) may also be obtained from PromoCell GmbH, Heidelberg, Germany, for investigating the biological effectiveness. The cells are then cultivated in Ham's F12 medium, supplemented with 10% fetal calf serum, 20 U/ml penicillin-streptomycin, 50 μg/ml partricin and 0.15 mg/ml glutamine.

To investigate the biological activity, monolayer cell cultures of the isolated human osteoblasts are incubated for a period of 24 hours in a medium that is supplemented with 0.5 mg/ml of the respective collagen peptide. A control is incubated in each case in a medium without peptide. The respective mRNA expression is then determined.

### Example 4—Skin Health

The stimulation of the synthesis of collagen (type I) and the proteoglycans biglycan and versican is investigated in vitro on human dermal fibroblasts (skin cells). For this purpose, the cells are incubated for 24 hours with 0.5 mg/ml of a low molecular weight or the collagen peptide according to the invention, and the expression of collagen RNA, biglycan RNA and versican RNA is then determined by real-time PCR and semi-quantitatively (based on a control without peptide).

### Example 5—Cartilage Health

For the cell cultures, porcine or human chondrocytes are isolated from cartilage tissue in a known manner and sown on culture plates at a density of approximately 350,000 cells/cm². Ham's F12 medium with 10% fetal calf serum, 10 μg/ml gentamicin and 5 μg/ml amphotericin B is used as the culture medium. As an alternative to 10 μg/ml gentamicin, 10 μg/ml penicillin-streptomycin may also be used. The cultivation took place at 37° C. in an oxygen-reduced atmosphere (5% O₂, 5% CO₂ and 90% N₂).

Determination of Collagen Biosynthesis:

The quantification of the collagen synthesized by the chondrocytes (essentially type II) is carried out by radioactive labeling with ¹⁴C-proline, which is incorporated into the collagen.

¹⁴C-proline is first added to the culture medium and the chondrocytes are cultivated under these conditions until the time of the determination. In order to be able to distinguish the incorporated from non-incorporated ¹⁴C-proline during the detection, the isotope-containing culture medium is then replaced by pure culture medium for a period of 3 days. The culture medium is then discarded and the adherent cell layer is mixed with distilled water in order to destroy the cell membranes through osmotic stress and to release cytosolic, unbounded ¹⁴C-proline. The cell debris with the synthesized extracellular matrix is pelleted by centrifugation. The pellet is re-suspended in fresh distilled water and a xylene scintillation cocktail is added. The amount of synthesized collagen may then be quantified by detecting the ¹⁴C-Proline with a beta counter.

Alternatively, the quantification may be carried out using the Sircol Collagen Assay Kit (Article No. 054S5000, 2019, tebu-bio, Offenbach, Germany, or Biocolor Ltd., UK) according to manufacturer instructions (see Example 6).

Determination of Proteoglycan Biosynthesis:

The proteoglycans synthesized by the chondrocytes are quantified by means of Alcian blue staining and photometric determination of the glycosaminoglycans (GAG), which are components of the proteoglycans.

In order to determine the GAG content in the cell culture, the culture medium is first discarded and the adherent cell layer is rinsed with PBS buffer (pH 7). The cells are then fixed in a 10% formaldehyde solution in PBS at 4° C. for 2 hours. After removing the formaldehyde, the Alcian blue staining reagent (5% Alcian blue in 3% acetic acid) is applied to the cell lawn and incubated at 4° C. overnight. Unbound Alcian blue is discarded and washed out by carefully rinsing three to four times with PBS. By adding acidic guanidine solution (8 mol/1), the GAG complexes are released from the cell layer. The amount of glycosaminoglycans may then be quantified photometrically at a wavelength of 620 nm.

Alternatively, the quantification may be carried out using the Blyscan Glycosaminoglycan Assay Kit (Article No. 054B3000, 2019, tebu-bio, Offenbach, Germany, or Biocolor Ltd., UK) according to manufacturer instructions (see Example 6).

### Example 6—Influence of Synthetic Collagen Peptides According to the Invention on the Biosynthesis of Matrix Proteins

Cell Culture:

The human cells used were obtained from tebu-bio GmbH, Offenbach, Germany. The chondrocytes (Cat. No. 402-05a) or dermal fibroblasts (Cat. No. 106-05a) were first sown in 12-well culture plates and cultured at 37° C., 5% CO₂ in Ham's F12 medium, to which 10% fetal calf serum, 20 U/ml penicillin-streptomycin and 50 μg/ml ascorbic acid were added. On every other day, the culture medium was replaced by new culture medium until the cells had reached 80% confluence. To investigate the influence of the collagen peptides according to the invention of SEQ ID No. 2 (non-hydroxylated), SEQ ID No. 7 (hydroxylated), SEQ ID No. 8 (hydroxylated), SEQ ID No. 9 (hydroxylated), SEQ ID No. 10 (hydroxylated), SEQ ID No. 11 (non-hydroxylated), SEQ ID No. 12 (non-hydroxylated), SEQ ID No. 13 (non-hydroxylated), SEQ ID No. 14 (non-hydroxylated), SEQ ID No. 15 (non-hydroxylated), SEQ ID No. 16 (non-hydroxylated), SEQ ID No. 17 (hydroxylated), SEQ ID No. 18 (hydroxylated), SEQ ID No. 19 (hydroxylated), SEQ ID No. 20 (hydroxylated), SEQ ID No. 21 (hydroxylated), SEQ ID No. 22 (hydroxylated), SEQ ID No. 23 (hydroxylated), SEQ ID No. 24 (hydroxylated), SEQ ID No. 25 (hydroxylated), SEQ ID No. 26 (hydroxylated), SEQ ID No. 27 (hydroxylated), SEQ ID No. 28 (hydroxylated), SEQ ID No. 29 (hydroxylated) and SEQ ID No. 30 (hydroxylated) on the biosynthesis of matrix proteins, the cell culture medium was then replaced by a special stimulation medium to which 0.5 mg/ml of the specific collagen peptides were added.

Collagen Assay:

To investigate the collagen metabolism, the amount of newly synthesized collagen was determined after stimulating the cells with 0.5 mg/ml BCP for three weeks. The synthesized collagen was isolated using the Sircol assay (Article No. 054S5000, 2019, tebu-bio, Offenbach, Germany, or Biocolor Ltd., UK) according to the manufacturer's instructions. In brief, the culture medium was initially discarded and the adherent cell layers were digested with 0.1 mg pepsin solution in 0.5 M acetic acid at 4° C. overnight. The cell suspensions were neutralized by adding 100 μl of an acid-neutralizing reagent. The synthesized collagen was then separated off by adding 200 μl of an isolation and concentration solution with vigorous shaking at 4° C. overnight. After centrifugation (12,000 rpm, 10 min) and discarding the supernatant, the isolated collagen was resuspended in 1 ml of Sircol dye solution. After 30 minutes of shaking and another centrifugation, the collagen pellet was covered with 750 μl of cold acidic solid washing reagent. After another centrifugation, the supernatant was again discarded and the enriched collagen was taken up in 250 μl of alkaline solution. In each case 200 μl of the sample solutions were used for photometric quantification of the synthesized collagen. The absorbance was measured at a wavelength of 492 nm. The amount of collagen synthesized in each case was determined on the basis of standardized collagen solutions.

An increased collagen synthesis by dermal fibroblasts compared to untreated controls could be found, which were incubated with the hydroxylated collagen peptides according to the invention of SEQ ID No. 7 (42% increase), SEQ ID No. 8 (37% increase), SEQ ID No. 9 (38% increase), SEQ ID No. 10 (35% increase), SEQ ID No. 17 (42% increase), SEQ ID No. 18 (40% increase), SEQ ID No. 19 (40% increase), SEQ ID No. 20 (37% increase), SEQ ID No. 21 (36% increase), SEQ ID No. 22 (43% increase), SEQ ID No. 23 (39% increase), SEQ ID No. 24 (36% increase), SEQ ID No. 25 (27% increase), SEQ ID No. 26 (12% increase), SEQ ID No. 27 (27% increase), SEQ ID No. 28 (22% increase), SEQ ID No. 29 (21% increase) and SEQ ID No. 30 (21% increase) (FIG. 1A; FIG. 4; FIG. 7A). It was also shown that the incubation of dermal fibroblasts with non-hydroxylated collagen peptides according to the invention causes at least a comparable stimulation of collagen synthesis as the incubation of the cells with a mixture of hydroxylated collagen peptides obtained from natural sources with a comparable average molecular weight (Verisol) (SEQ ID No. 2 (22% increase), SEQ ID No. 11 (19% increase), SEQ ID No. 12 (22% increase), SEQ ID No. 13 (20% increase), SEQ ID No. 14 (22% increase), SEQ ID No. 15 (23% increase) and SEQ ID No. 16 (22% increase)). All examined collagen peptides according to the invention showed a significant increase in collagen synthesis compared to gelatin controls (Gelatin 160, Gelatin 260).

An increased collagen synthesis could also be observed for chondrocytes which were incubated with the collagen peptides according to the invention compared to untreated controls. The incubation of chondrocytes with hydroxylated collagen peptides led to an increase in collagen synthesis by 28% (SEQ ID No. 7), 24% (SEQ ID Nos. 8, 9), 20% (SEQ ID No. 10), 33% (SEQ ID No. 17), 32% (SEQ ID No. 18), 37% (SEQ ID No. 19), 30% (SEQ ID No. 20), 55% (SEQ ID No. 21), 46% (SEQ ID No. 22), 19% (SEQ ID Nos. 23, 25), 26% (SEQ ID No. 24), and 12% (SEQ ID No. 26). The incubation of chondrocytes with non-hydroxylated collagen peptides according to the invention led to at least a comparable stimulation of collagen synthesis as the incubation of cells with a mixture of hydroxylated collagen peptides obtained from natural sources with a comparable average molecular weight (Fortigel) (FIG. 1B; FIG. 3). The incubation of chondrocytes with non-hydroxylated collagen peptides led to an increase in collagen synthesis by 16% (SEQ ID No. 2, 11), 24% (SEQ ID No. 12), 15% (SEQ ID No. 13), 28% (SEQ ID No. 14) and 31% (SEQ ID Nos. 15, 16).

Proteoglycan Assay:

The Blyscan glycosaminoglycan assay (Article No. 054B3000, 2019, tebu-bio, Offenbach, Germany, or Biocolor Ltd., UK) was used to determine proteoglycans. The biosynthesis of proteoglycans was determined after stimulating dermal fibroblasts for two weeks. According to the manufacturer's instructions, after discarding the culture medium, the cell layers were covered with 1 ml of papain extraction solution and incubated for three hours at 65° C. with vigorous shaking. The cell suspensions were then centrifuged (10,000 g, 10 min) and the supernatants were collected. After adding 1 ml of Blyscan dye solution and shaking (30 min), the supernatants were centrifuged again (12,000 rpm, 10 min) and then discarded. The isolated proteoglycan pellets were resuspended in 500 μl of dissociation solution. A photometric determination of the synthesized proteoglycan in 200 μl sample solution was carried out at a wavelength of 656 nm in comparison to untreated control experiments.

As shown in FIGS. 2A, 6 and 7B, the synthesis of proteoglycans by dermal fibroblasts was significantly increased in comparison to untreated controls when the fibroblasts were incubated with collagen peptides according to the invention. Incubation of the cells with hydroxylated collagen peptides led to an increase in the synthesis of proteoglycans by 41% (SEQ ID No. 7), 23% (SEQ ID No. 8, 25), 27% (SEQ ID No. 9, 21), 25% (SEQ ID No. 10), 24% (SEQ ID No. 17), 31% (SEQ ID No 18), 29% (SEQ ID Nos. 19, 20), 28% (SEQ ID No. 22), 22% (SEQ ID No. 23, 28, 30), 26% (SEQ ID No. 24, 27), 6% (SEQ ID No. 26) and 21% (SEQ ID No. 29). It could also be shown that incubation of dermal fibroblasts with non-hydroxylated collagen peptides according to the invention leads to at least a comparable stimulation of the synthesis of proteoglycans as when the cells are incubated with a mixture of hydroxylated collagen peptides obtained from natural sources with a comparable average molecular weight (Verisol). The incubation of dermal fibroblasts with non-hydroxylated collagen peptides led to an increase in collagen synthesis by 18% (SEQ ID No. 2, 11, 15, 16), 19% (SEQ ID No. 12), 16% (SEQ ID No. 13) and 14% (SEQ ID No. 14). A significant increase in the synthesis of proteoglycans compared to gelatin controls (Gelatin 160, Gelatin 260) could be observed for all of the examined collagen peptides according to the invention.

Chondrocytes which were incubated with the collagen peptides according to the invention also show an increased synthesis of proteoglycans compared to untreated controls (FIGS. 2B and 5). The incubation of chondrocytes with hydroxylated collagen peptides led to an increase in collagen synthesis by 30% (SEQ ID No. 7), 20% (SEQ ID No. 8), 19% (SEQ ID No. 9), 29% (SEQ ID No. 10), 31% (SEQ ID No. 17), 25% (SEQ ID No. 18), 18% (SEQ ID No. 19), 15% (SEQ ID No. 20, 24), 22% (SEQ ID No. 21), 26% (SEQ ID No. 22, 24) and 11% (SEQ ID No. 23). The incubation of chondrocytes with non-hydroxylated collagen peptides according to the invention led to at least a comparable stimulation of the synthesis of proteoglycans as the incubation of cells with a mixture of hydroxylated collagen peptides obtained from natural sources with a comparable average molecular weight (Fortigel) (FIG. 5; FIG. 2B). The incubation of chondrocytes with non-hydroxylated collagen peptides led to an increase in the synthesis of proteoglycans by 22% (SEQ ID No. 2), 28% (SEQ ID No. 11), 23% (SEQ ID No. 12), 9% (SEQ ID No. 13), 14% (SEQ ID No. 14), 6% (SEQ ID No. 15) and 18% (SEQ ID No. 16).

1-20. (canceled)

21. A synthetic or recombinant collagen peptide having a molecular weight in a range from 0.18 to 10.0 kDa, in particular 0.18 to 5.0 kDa, in particular 1.1 to 5.0 kDa.

22. The collagen peptide according to claim 21, wherein the collagen peptide has the amino acid sequence SEQ ID No. 1, 15 or a function-preserving sequence modification thereof.

23. The collagen peptide according to claim 21, wherein the collagen peptide has the amino acid sequence SEQ ID No. 1, in which at least one proline residue is hydroxylated, in particular wherein the collagen peptide has the amino acid sequence SEQ ID No. 6.

24. The collagen peptide according to claim 21, wherein the collagen peptide has an amino acid sequence selected from the group consisting of SEQ ID No. 1 to 30, in particular selected from the group consisting of SEQ ID No. 1 to 4, 6 to 9, 12 to 14, and 18 to 20.

25. The collagen peptide according to claim 21, wherein the collagen peptide is able to stimulate the synthesis of extracellular matrix proteins, in particular collagen, in connective tissue cells, in particular in osteoblasts, chondrocytes and/or fibroblasts.

26. The collagen peptide according to claim 21, wherein the collagen peptide has no amino acid modification, in particular has no hydroxylation.

27. The collagen peptide according to claim 21, wherein the collagen peptide comprises hydroxylated proline, hydroxylated lysine, or hydroxylated proline and lysine.

28. The collagen peptide according to claim 21, wherein the collagen peptide is glycosylated on at least one hydroxylated lysine.

29. The collagen peptide according to claim 21 to be applied in a method for maintaining and improving bone health, for preventing and/or treating osteoporosis, for preventing and/or treating sarcopenia, for preventing and/or treating degenerative loss of muscle mass, for preventing and/or treating cartilage diseases, for improving muscle strength, for preventing and/or treating a pathological condition characterized by reduced mitochondrial activity, in particular for preventing and/or treating a pathological condition characterized by reduced endurance, for stimulating fat loss, for reducing body weight and/or for preventing and/or treating degenerative joint diseases, for preventing and/or treating diseases of the tendons and ligaments, for preventing and/or treating skin diseases, for preventing and/or treating degenerative nerve diseases, for preventing and/or treating dementia, for preventing and/or treating Alzheimer's disease, for preventing and/or treating a pathological condition characterized by a reduction in mental performance, for preventing and/or treating diseases associated with dysfunction of the blood-brain barrier, for preventing and/or treating intestinal diseases, in particular chronically inflammable intestinal diseases, for preventing and/or treating diseases of the cardiovascular system, in particular of the structure and/or function of the blood vessels or for preventing or treating periodontal diseases.

30. A pharmaceutical preparation, comprising a collagen peptide according to claim 21 and at least one pharmaceutically acceptable additive, in particular to be applied in a method for preventing and/or treating bone diseases, in particular osteoporosis, for preventing and/or treating sarcopenia, for preventing and/or treating degenerative loss of muscle mass, for preventing and/or treating cartilage diseases, for improving muscle strength, for stimulating fat loss, for reducing body weight, for preventing and/or treating degenerative joint diseases, for preventing and/or treating diseases of the tendons and ligaments, for preventing and/or treating skin diseases, for preventing and/or treating degenerative nerve diseases, for preventing and/or treating dementia, for preventing and/or treating Alzheimer's disease, for preventing and/or treating diseases associated with dysfunction of the blood-brain barrier, for preventing and/or treating intestinal diseases, in particular chronically inflammable intestinal diseases, for preventing and/or treating diseases of the cardiovascular system, in particular of the structure and/or function of the blood vessels or for preventing and/or treating periodontal diseases.

31. A food supplement, food or luxury food comprising a collagen peptide according to claim 21 and at least one food-acceptable additive.

32. A cosmetic product comprising a collagen peptide according to claim 21 and at least one skin-compatible additive.

33. A method for improving muscle strength, for increasing muscle mass, for stimulating fat loss, for reducing body weight, for maintaining and/or improving bone health, for maintaining and/or improving skin health, for maintaining and/or improving the intestinal health, for maintaining and/or improving the blood vessel structure, for maintaining and/or improving the health of the cardiovascular system, for maintaining and/or improving the gums, for maintaining and/or improving the health of the nails and hair of a human or animal body, for maintaining and/or increasing the number of mitochondria and/or mitochondrial activity, for maintaining and/or improving endurance performance or for maintaining and/or improving mental performance, comprising administering at least one collagen peptide preparation according to claim 21 to a human or animal body.

34. A method for the visual and structural improvement of the skin, in particular to reduce the formation of wrinkles, improve the skin elasticity, increase the resilience of the skin, increase the moisture content of the skin, reduce cellulite and/or reduce stretch marks, especially stretch marks from pregnancy, comprising administering at least one synthetic or recombinant collagen peptide according to claim 21 to a human or animal body.

35. A method for accelerating the growth of the nails and/or for reducing the fragility of nails, comprising administering at least one synthetic or recombinant collagen peptide according to claim 21 to a human or animal body.

36. A method for the visual and structural improvement of the hair, in particular for improving the hair quality, reducing split ends and/or reducing or delaying hair loss, comprising administering at least one synthetic or recombinant collagen peptide according to claim 21 to a human or animal body.

37. A method for the recombinant production of a collagen peptide according to claim 21, comprising the method steps:
a) providing an expression system that has at least one expression cassette, wherein the expression cassette has at least one nucleotide sequence that encodes a collagen peptide having a molecular weight in a range from 0.18 to 10.0 kDa, in particular 0.18 to 5.0 kDa, in particular 1.1 to 5.0 kDa,
b) cultivating the expression system under conditions that allow expression of the collagen peptide, and
c) obtaining the collagen peptide.

38. A method according to claim 37, wherein the expression system is a host cell selected from the group consisting of bacterial cell, yeast cell, fungal cell, mammalian cell, insect cell, and plant cell.

39. The method according to claim 37, wherein the collagen peptide encoded by the nucleotide sequence is a collagen peptide of a vertebrate, in particular a mammal, a bird, a fish, an amphibian, a reptile or an invertebrate animal.

40. The method according to claim 37, wherein the expression system is a host cell, which is capable of hydroxylating proline residues, lysine residues or proline residues and lysine residues of the expressed collagen peptide.

41. The method according to claim 37, wherein the host cell includes at least one expression cassette which includes a polynucleotide sequence that encodes a prolyl 4-hydroxylase, and wherein an in vivo hydroxylated collagen peptide is obtained.

42. The method according to claim 37, wherein the host cell has at least one expression cassette which includes a lysyl hydroxylase-coding polynucleotide sequence, and wherein an in vivo hydroxylated collagen peptide is obtained.

43. The method according to claim 37, wherein the expression system is not capable of causing hydroxylation of proline residues, lysine residues or proline and lysine residues of the expressed collagen peptide.
